(19) United States
(12) Patent Application Publication
(10) Pub. No.: US 2024/0324620 A1
(43) Pub. Date: Oct. 3, 2024

(54) COMPOSITIONS AND METHODS FOR PRODUCING FERMENTED DAIRY COMPOSITIONS HAVING CREAM FLAVOR

(71) Applicant: DUPONT NUTRITION BIOSCIENCES APS, Kgs. Lyngby (DK)

(72) Inventors: Damien Lugand, Neuilly-sur-Seine (FR); Elise Manoury, Chatellerault (FR); Sandrine Gomez, Coussay-Les-Bois (FR)

(21) Appl. No.: 18/580,967

(22) PCT Filed: Jul. 28, 2022
(86) PCT No.: PCT/EP2022/071206

§ 371 Date: Jan. 19, 2024

Related U.S. Application Data
(60) Provisional application No. 63/226,958, filed on Jul. 29, 2021.

(30) Foreign Application Priority Data
Nov. 4, 2021 (EP) 21206399.4

(51) Int. Cl. A23C 9/127; C12Q 1/04

(52) U.S. Cl. CPC A23C 9/127; C12Q 1/04

(57) ABSTRACT

Provided herein are compositions and methods useful for producing thermophilic fermented dairy compositions, such as thermophilic fermented dairy products, having cream flavor. The compositions and methods provided herein include bacteria, e.g., Lacticaseibacillus rhamnosus, useful for decreasing acetaldehyde content and increasing diacetyl content in a thermophilic fermented dairy composition and producing a cream flavor. Thermophilic fermented dairy compositions, including stored thermophilic fermented dairy compositions, produced according to the methods described herein are provided. Also provided are methods of identifying bacteria useful for producing thermophilic fermented dairy compositions having cream flavor.

### FIELD OF THE INVENTION

Provided herein are compositions and methods useful for producing thermophilic fermented dairy compositions, such as thermophilic fermented dairy products, having cream flavor. The compositions and methods provided herein include bacteria, e.g., Lacticaseibacillus rhamnosus, useful for decreasing acetaldehyde content and increasing diacetyl content in a thermophilic fermented dairy composition and producing a cream flavor. Thermophilic fermented dairy compositions, including stored thermophilic fermented dairy compositions, produced according to the methods described herein are provided. Also provided are methods of identifying bacteria useful for producing thermophilic fermented dairy compositions having cream flavor, such as in the screening for such bacteria.

### BACKGROUND TO THE INVENTION

Flavor perception is a complex sensory phenomenon that contributes to the acceptance and desirability of food products by consumers. In fermented dairy products, e.g., thermophilic fermented dairy products, such as yogurts, creams, milk beverages, and cheeses, cream flavor is an important flavor signature for consumer acceptance. Cream flavor perception is particularly important for fat free and reduced fat fermented dairy products, which often lack cream flavor.

Thus, there exists a need for compositions and methods for naturally producing and improving cream flavor in fermented dairy compositions, such as thermophilic fermented dairy products. The compositions and methods provided herein address such needs.

### SUMMARY OF THE INVENTION

In aspects are provide methods for producing a thermophilic fermented dairy composition having a cream flavor, the methods including decreasing acetaldehyde content and increasing diacetyl content in the thermophilic fermented dairy composition. Also provided herein are methods for producing a stored thermophilic fermented dairy composition having a cream flavor, the methods including: (a) storing a thermophilic fermented dairy composition to produce the stored thermophilic fermented dairy composition; and (b) decreasing acetaldehyde content and increasing diacetyl content in the thermophilic fermented dairy composition during storage. In some embodiments, the methods further include producing the thermophilic fermented dairy composition, the producing including fermenting a milk substrate inoculated with a starter culture to produce the thermophilic fermented dairy composition.

In aspects are provided methods for producing a thermophilic fermented dairy composition having a cream flavor, the methods including: (a) fermenting a milk substrate inoculated with a starter culture to produce a thermophilic fermented dairy composition; and (b) decreasing acetaldehyde content and increasing diacetyl content in the thermophilic fermented dairy composition. In some embodiments, the methods include storing the thermophilic fermented dairy composition to produce a stored thermophilic fermented dairy composition.

In some embodiments, the acetaldehyde content is decreased and the diacetyl content is increased in the thermophilic fermented dairy composition during storage. In some embodiments, (a) the acetaldehyde content is decreased and the diacetyl content is increased to produce a ratio of acetaldehyde to diacetyl that is less than 1, 0.75, 0.5, 0.25, 0.1, 0.075, 0.05, 0.025, or 0.01, or is 0; and (b) the diacetyl content is increased to at least 1 ppm. In some embodiments, a) the diacetyl content is increased to about 1 ppm, about 5 ppm, about 10 ppm, or about 15 ppm; and b) the acetaldehyde content is decreased to produce a ratio of acetaldehyde to diacetyl that is less than about 1, 0.75, 0.5, 0.25, 0.1, 0.075, 0.05, 0.025, or 0.01, or is 0. In some embodiments, (a) the acetaldehyde content is decreased to below about 1 ppm and the diacetyl content is increased to above about 1.5 ppm; or (b) the acetaldehyde content is decreased to below about 0.2 ppm, and the diacetyl content is increased to above about 5 ppm.

In some embodiments, the starter culture contains one or more lactic acid bacteria. In some embodiments, the starter culture includes bacteria from the genus Lactococcus, Lactobacillus, Streptococcus, Lacticaseibacillus, Leuconostoc, Pediococcus, Bifidobacterium, or any combination of the foregoing. In some embodiments, the starter culture contains one or more Lacticaseibacillus rhamnosus strains. In some embodiments, the one or more Lacticaseibacillus rhamnosus strains include or are: (a) the Lacticaseibacillus rhamnosus strain deposited under accession number DSM33650 at the DSMZ or a mutant thereof; (b) the Lacticaseibacillus rhamnosus strain deposited under accession number DSM22876 at the DSMZ or a mutant thereof; and/or (c) the Lacticaseibacillus rhamnosus strain deposited under accession number DSM22193 at the DSMZ or a mutant thereof. In some embodiments, the starter culture contains one or more Streptococcus thermophilus strains. In some embodiments, the one or more Streptococcus thermophilus strains include or are: (a) the Streptococcus thermophilus strain deposited under accession number DSM33651 at the DSMZ or a mutant thereof; (b) the Streptococcus thermophilus strain deposited under accession number DSM33849 at the DSMZ or a mutant thereof; and/or (c) the Streptococcus thermophilus strain deposited under accession number DSM33829 at the DSMZ or a mutant thereof. In some embodiments, the starter culture includes or is: (a) the Lacticaseibacillus rhamnosus strain deposited under accession number DSM 33650 at the DSMZ or a mutant thereof and the Streptococcus thermophilus strain deposited under accession number DSM 33651 at the DSMZ or a mutant thereof; (b) the Lacticaseibacillus rhamnosus strain deposited under accession number DSM33650 at the DSMZ or a mutant thereof and the Streptococcus thermophilus strain deposited under accession number DSM33849 at the DSMZ or a mutant thereof; (c) the Lacticaseibacillus rhamnosus strain deposited under accession number DSM33650 at the DSMZ or a mutant thereof and the Streptococcus thermophilus strain deposited under accession number DSM33829 at the DSMZ or a mutant thereof; (d) the Lacticaseibacillus rhamnosus strain deposited under accession number DSM22876 at the DSMZ or a mutant thereof and the Streptococcus thermophilus strain deposited under accession number DSM33651 at the DSMZ or a mutant thereof; (e) the Lacticaseibacillus rhamnosus strain deposited under accession number DSM22876 at the DSMZ or a mutant thereof and the Streptococcus thermophilus strain deposited under accession number DSM33849 at the DSMZ or a mutant thereof; (f) the Lacticaseibacillus rhamnosus strain deposited under accession number DSM22876 at the DSMZ or a mutant thereof and the Streptococcus thermophilus strain deposited under accession number DSM33829 at the DSMZ or a mutant thereof; (g) the Lacticaseibacillus rhamnosus strain deposited under accession number DSM 22193 at the DSMZ or a mutant thereof and the Streptococcus thermophilus strain deposited under accession number DSM33651 at the DSMZ or a mutant thereof; (h) the Lacticaseibacillus rhamnosus strain deposited under accession number DSM22193 at the DSMZ or a mutant thereof and the Streptococcus thermophilus strain deposited under accession number DSM33849 at the DSMZ or a mutant thereof; or (i) the Lacticaseibacillus rhamnosus strain deposited under accession number DSM22193 at the DSMZ or a mutant thereof and the Streptococcus thermophilus strain deposited under accession number DSM33829 at the DSMZ or a mutant thereof. In some embodiments, the starter culture contains one or more of a Lactobacillus delbrueckii subsp bulgaricus strain, a Lactobacillus acidophilus strain, a Bifidobacterium lactis strain, Limosilactobacillus fermentum strain, a Lacticaseibacillus paracasei strain, a Lactiplantibacillus plantarum strain, a Propionibacteria freudenreichii strain, a Pediococcus acidilactici strain, an a Lactococcus cremoris subsp cremoris strain, or a Lactococcus lactis subsp lactis strain. In some embodiments, the milk substrate is fermented at a temperature above about 33° C., 34° C., 35° C., 36° C., 37° C., 38° C., 39° C., 40° C., 41° C., 42° C., 43° C., 44° C., 45° C., or 50° C. In some embodiments, the thermophilic fermented dairy composition has a pH of about 3.8 to about 5. In some embodiments, the thermophilic fermented dairy composition has a pH of about 4.2 to about 4.6. In some embodiments, the thermophilic fermented dairy composition is stored for: a) at least about 1, 2, 3, 4, 5, 6, 7, 8, 9, 10, 11, 12, 13, 1415, 16, 17, 18, 19, 20, 21, 22, 23, 24, 25, 26, 27, 28, 29, 30, 40, 50, or about 60 days; or b) at most about 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 40, 50, or about 60 days. In some embodiments, the thermophilic fermented dairy composition is stored for about 28 days at about 6° C. In some embodiments, the thermophilic fermented dairy composition is stored for about 14 days at about 6° C. In some embodiments, the stored thermophilic fermented dairy composition has a pH of about 3.8 to about 5. In some embodiments, the stored thermophilic fermented dairy composition has a pH of about 4.5 or about 4.6.

In some embodiments, the milk substrate is cow milk. In some embodiments, the cream flavor includes a cream aroma and/or a cream odor. In some embodiments, the cream flavor is an increased cream flavor compared to a cream flavor of a thermophilic fermented dairy composition having an acetaldehyde content and a diacetyl content resulting in a ratio of acetaldehyde to diacetyl of equal to or greater than 1. In some embodiments, the increased cream flavor includes an increased cream aroma and/or cream odor compared to a cream aroma and/or cream odor of a thermophilic fermented dairy composition having an acetaldehyde content and a diacetyl content resulting in a ratio of acetaldehyde to diacetyl of equal to or greater than 1.

In aspects are provided compositions containing a Lacticaseibacillus rhamnosus strain, wherein the Lacticaseibacillus rhamnosus strain decreases acetaldehyde and increases diacetyl in a thermophilic fermented dairy composition to produce a cream flavor. In some embodiments, the Lacticaseibacillus rhamnosus strain is: (a) the Lacticaseibacillus rhamnosus strain deposited under accession number DSM33650 at the DSMZ or a mutant thereof; (b) the Lacticaseibacillus rhamnosus strain deposited under accession number DSM22876 at the DSMZ or a mutant thereof; or (c) the Lacticaseibacillus rhamnosus strain deposited under accession number DSM22193 at the DSMZ or a mutant thereof. In some embodiments, the composition further contains a Streptococcus thermophilus strain. In some embodiments, the Streptococcus thermophilus strains is: (a) the Streptococcus thermophilus strain deposited under accession number DSM33651 at the DSMZ or a mutant thereof; (b) the Streptococcus thermophilus strain deposited under accession number DSM33849 at the DSMZ or a mutant thereof; and/or (c) the Streptococcus thermophilus strain deposited under accession number DSM33829 at the DSMZ or a mutant thereof. In some embodiments, the composition contains or is: (a) the Lacticaseibacillus rhamnosus strain deposited under accession number DSM33650 at the DSMZ or a mutant thereof and the Streptococcus thermophilus strain deposited under accession number DSM33651 at the DSMZ or a mutant thereof; (b) the Lacticaseibacillus rhamnosus strain deposited under accession number DSM33650 at the DSMZ or a mutant thereof and the Streptococcus thermophilus strain deposited under accession number DSM33849 at the DSMZ or a mutant thereof; (c) the Lacticaseibacillus rhamnosus strain deposited under accession number DSM33650 at the DSMZ or a mutant thereof and the Streptococcus thermophilus strain deposited under accession number DSM33829 at the DSMZ or a mutant thereof; (d) the Lacticaseibacillus rhamnosus strain deposited under accession number DSM22876 at the DSMZ or a mutant thereof and the Streptococcus thermophilus strain deposited under accession number DSM33651 at the DSMZ or a mutant thereof; (e) the Lacticaseibacillus rhamnosus strain deposited under accession number DSM22876 at the DSMZ or a mutant thereof and the Streptococcus thermophilus strain deposited under accession number DSM33849 at the DSMZ or a mutant thereof; (f) the Lacticaseibacillus rhamnosus strain deposited under accession number DSM22876 at the DSMZ or a mutant thereof and the Streptococcus thermophilus strain deposited under accession number DSM33829 at the DSMZ or a mutant thereof; (g) the Lacticaseibacillus rhamnosus strain deposited under accession number DSM22193 at the DSMZ or a mutant thereof and the Streptococcus thermophilus strain deposited under accession number DSM33651 at the DSMZ or a mutant thereof; (h) the Lacticaseibacillus rhamnosus strain deposited under accession number DSM22193 at the DSMZ or a mutant thereof and the Streptococcus thermophilus strain deposited under accession number DSM33849 at the DSMZ or a mutant thereof; or (i) the Lacticaseibacillus rhamnosus strain deposited under accession number DSM22193 at the DSMZ or a mutant thereof and the Streptococcus thermophilus strain deposited under accession number DSM33829 at the DSMZ or a mutant thereof. In some embodiments, the composition further includes one or more of a Lactobacillus delbrueckii subsp bulgaricus strain, a Lactobacillus acidophilus strain, a Bifidobacterium lactis strain, Limosilactobacillus fermentum strain, a Lacticaseibacillus paracasei strain, a Lactiplantibacillus plantarum strain, a Propionibacteria freudenreichii strain, a Pediococcus acidilactici strain, a Lactococcus cremoris subsp cremoris strain, or a Lactococcus lactis subsp lactis strain. In some embodiments, the composition is a starter culture. In some embodiments, the composition is a thermophilic fermented dairy product.

In aspects are provided uses of Lacticaseibacillus rhamnosus to decrease acetaldehyde content and increase diacetyl content in a thermophilic fermented dairy composition to produce cream flavor. In some embodiments, the Lacticaseibacillus rhamnosus is: (a) the Lacticaseibacillus rhamnosus strain deposited under accession number DSM33650 at the DSMZ or a mutant thereof; (b) the Lacticaseibacillus rhamnosus strain deposited under accession number DSM22876 at the DSMZ or a mutant thereof; or (c) a Lacticaseibacillus rhamnosus strain deposited under accession number DSM22193 at the DSMZ or a mutant thereof.

In some embodiments, the compositions and uses produce a cream flavor. In some embodiments, the cream flavor includes a cream aroma and/or a cream odor. In some embodiments, the cream flavor is an increased cream flavor compared to a cream flavor of a thermophilic fermented dairy composition having an acetaldehyde content and a diacetyl content resulting in a ratio of acetaldehyde to diacetyl of equal to or greater than 1. In some embodiments, the increased cream flavor includes an increased cream aroma and/or cream odor compared to a cream aroma and/or cream odor of a thermophilic fermented dairy composition having an acetaldehyde content and a diacetyl content resulting in a ratio of acetaldehyde to diacetyl of equal to or greater than 1.

In aspects are provided thermophilic fermented dairy compositions obtained according to any of the methods or uses described herein. In some embodiments, the thermophilic fermented dairy composition is a thermophilic fermented dairy product. In some embodiments, the thermophilic fermented dairy composition is a yogurt, a cream, a matured cream, a cheese, fromage frais, a milk beverage, a processed cheese, a cream dessert, a cottage cheese, an infant milk, or a kefir.

In aspects are provided thermophilic fermented dairy products having cream flavor, the thermophilic fermented dairy product containing a ratio of acetaldehyde content to diacetyl content that is less than 1, 0.75, 0.5, 0.25, 0.1, 0.075, 0.05, 0.025, or 0.01, or is 0. In some embodiments, the thermophilic fermented dairy product includes a Lacticaseibacillus rhamnosus strain that decreases acetaldehyde and increases diacetyl content in the thermophilic fermented dairy product. In some embodiments, the Lacticaseibacillus rhamnosus strain is: (a) a Lacticaseibacillus rhamnosus strain deposited under accession number DSM33650 or a mutant thereof; (b) a Lacticaseibacillus rhamnosus strain deposited under accession number DSM22876 or a mutant thereof; and/or (c) a Lacticaseibacillus rhamnosus strain deposited under accession number DSM22193 or a mutant thereof. In some embodiments, the thermophilic fermented dairy products further include: (a) the Streptococcus thermophilus strain deposited under accession number DSM33651 at the DSMZ or a mutant thereof; (b) the Streptococcus thermophilus strain deposited under accession number DSM33849 at the DSMZ or a mutant thereof; and/or (c) the Streptococcus thermophilus strain deposited under accession number DSM33829 at the DSMZ or a mutant thereof. In some embodiments, the thermophilic fermented dairy product is a yogurt, a cream, a matured cream, a cheese, fromage frais, a milk beverage, a processed cheese, a cream dessert, a cottage cheese, an infant milk, or a kefir.

Also provided herein are methods of identifying one or more bacteria capable of producing a thermophilic fermented dairy composition having cream flavor, the methods including quantifying acetaldehyde content and diacetyl content in a thermophilic fermented dairy composition fermented in the presence of one or more bacteria. In some embodiments, the thermophilic fermented dairy composition is stored and the acetaldehyde and diacetyl content are quantified during storage. In some embodiments, if the quantified acetaldehyde content and diacetyl content produce a ratio of acetaldehyde content to diacetyl content in the thermophilic fermented dairy composition that is less than 1, 0.75, 0.5, 0.25, 0.1, 0.075, 0.05, 0.025, or 0.01, or is 0, the one or more bacteria are identified as capable of producing a thermophilic fermented dairy composition having cream flavor. In some embodiments, the one or more bacteria include lactic acid bacteria. In some embodiments, the one or more bacteria include a Lacticaseibacillus rhamnosus strain. In some embodiments, the one or more bacteria comprise a Streptococcus thermophilus strain.

In some embodiments, the ratio of acetaldehyde to diacetyl associated with cream flavor, e.g., increased cream flavor, as described herein is reached after 28 days or less in storage. In some embodiments, the ratio of acetaldehyde to diacetyl associated with cream flavor, e.g., increased cream flavor, as described herein is reached after 21 days or less in storage. In some embodiments, the ratio of acetaldehyde to diacetyl associated with cream flavor, e.g., increased cream flavor, as described herein is reached after 14 days or less in storage. In some embodiments, the ratio of acetaldehyde to diacetyl associated with cream flavor, e.g., increased cream flavor, as described herein is reached after 7 days or less in storage.

In aspects are provided compositions containing one or more bacteria identified as capable of producing a thermophilic fermented dairy composition having cream flavor according to the methods described herein. In some embodiments, the composition is a starter culture. In some embodiments, the composition is a thermophilic fermented dairy product.

Also provided herein are kits containing a composition as described herein and instructions for use.

In an aspect is provided a Streptococcus thermophilus strain deposited under accession number DSM33849 at the DSMZor a mutant thereof. In an aspect is provided a Streptococcus thermophilus strain deposited under accession number DSM33829 at the DSMZ or a mutant thereof. In an aspect is provided a Streptococcus thermophilus strain deposited under accession number DSM33651 at the DSMZ or a mutant thereof. In an aspect is provided a Lacticaseibacillus rhamnosus strain deposited under accession number DSM22876 at the DSMZ or a mutant thereof. In an aspect is provided a Lacticaseibacillus rhamnosus strain deposited underaccession number DSM22193 at the DSMZ or a mutant thereof.

Each of the aspects and embodiments described herein are capable of being used together, unless excluded either explicitly or clearly from the context of the embodiment or aspect.

### BRIEF DESCRIPTION OF THE FIGURES

FIGS. 1A-1B are spider plots showing the organoleptic profiles of plain full milks fermented with an exemplary Streptococcus thermophilus strain (DSM33849) alone or in combination with an exemplary L. rhamnosus strain (DSM22876, DSM33650, DSM22193, DSM24616, or industrial strain Strain 5) as indicated. Mean values are shown for 13 descriptors evaluated on a linear 7-point scale by a trained panel of sensory assessors. FIG. 1A shows organoleptic profiles of the fermented milks after one day in storage at 6° C. FIG. 1B shows organoleptic profiles of the fermented milks after 14 days in storage at 6° C.

FIGS. 2A-2C show the concentration of diacetyl (2,3-butanedione) and acetaldehyde (in ppm) in the fermented plain full milks described in FIGS. 1A-1B after one day (FIG. 2A), 14 days (FIG. 2B), and 28 days (FIG. 2C) in storage at 6° C.

FIGS. 3A-3B are spider plots showing the organoleptic profiles of diluted milks fermented with an exemplary Streptococcus thermophilus strain (DSM33849) alone or in combination with an exemplary L. rhamnosus strain (DSM22876, DSM33650, DSM22193, DSM24616, or industrial strain Strain 5) as indicated. Mean values are shown for 13 descriptors evaluated on a linear 7-point scale by a trained panel sensory assessors. FIG. 3A shows organoleptic profiles of the fermented milks after one day in storage at 6° C. FIG. 3B shows organoleptic profiles of the fermented milks after 14 days in storage at 6° C.

FIGS. 4A-4C show the concentration of diacetyl (2,3-butanedione) and acetaldehyde (in ppm) in the fermented diluted milks described in FIGS. 3A-3B after one day (FIG. 4A), 14 days (FIG. 4B), and 28 days (FIG. 4C) in storage at 6° C.

FIG. 5 is a scatter plot showing the concentration of diacetyl (2,3-butanedione) and acetaldehyde (in ppm) in the fermented plain full milks (Full) and diluted milks (Diluted) as described above after 14 days in storage at at 6° C. Also shown are diacetyl (2,3-butanedione) and acetaldehyde concentrations in the milks prior to fermentation (Milk D: diluted milk; Milk F: plain full milk).

FIG. 6 is a scatter plot showing the concentration of diacetyl (2,3-butanedione) and acetaldehyde (in ppm) after 1, 7, 14, 21, and 28 days in storage 6° C., for plain full milks fermented with exemplary S. thermophilus strains DSM33849, DSM33651, or DSM33829 alone or in combination with one of two exemplary Lacticaseibacillus rhamnosus strains: DSM33650 or DSM24616.

FIG. 7 is a spider plot showing the organoleptic profiles after 14 and 28 days in storage at 6° C. for plain full milks fermented with an exemplary Streptococcus thermophilus strain (DSM33651) alone or in combination with an exemplary L. rhamnosus strain (DSM33650 or DSM24616) as indicated. Mean values are shown for 13 descriptors evaluated on a linear 7-point scale by a trained panel of sensory assessors.

FIG. 8 is a PCA biplot showing the distance of the different fermented plain full milks (as described in FIG. 6) after 14 days in storage at 6° C. on the 13 sensory descriptors evaluated.

### DETAILED DESCRIPTION OF THE INVENTION

Provided herein are compositions and methods useful for producing thermophilic fermented dairy compositions, such as thermophilic fermented dairy products, having cream flavor. As used herein, a thermophilic fermented dairy composition or thermophilic fermented dairy product is a composition or product that is produced by thermophilic fermentation of a dairy milk substrate, for example as described herein. It was surprisingly found that thermophilic fermented dairy milks having a diacetyl (also referred to as 2,3-butanedione) content greater than the content of acetaldehyde had an increased cream flavor, e.g., cream aroma and/or cream odor, compared to thermophilic fermented dairy milks with about equal amounts of acetaldehyde and diacetyl or with an acetaldehyde content greater than a diacetyl content (see, e.g., Examples 1 and 2). Interestingly, this result was observed for both full fat and diluted thermophilic fermented dairy milks. Thus, the methods and compositions provided herein are capable of increasing cream flavor in thermophilic fermented dairy compositions having reduced fat content and/or reduced flavor compound precursors. As described herein, thermophilic fermented dairy compositions and products refer to dairy compositions and products that have undergone thermophilic fermentation such as described in Section I-C below.

While it has been previously appreciated that an increased concentration of diacetyl in fermented milk enhances cream flavor, it was not previously contemplated that an increase in diacetyl content combined with a decrease in acetaldehyde content would associate, e.g., correlate, with a cream flavor in thermophilic fermented dairy compositions, nor that a cream flavor correlated with a diacetyl content greater than its acetaldehyde content would be increased compared to thermophilic fermented dairy compositions produced to increase diacetyl content without decreasing levels of acetaldehyde. Thus, the methods and compositions provided herein harness the surprising findings described herein that thermophilic fermented dairy compositions, e.g., thermophilic fermented dairy products, having a diacetyl content greater than an acetaldehyde content have a cream flavor that is increased compared to cream flavor in thermophilic fermented dairy compositions having about equal levels of acetaldehyde and diacetyl or an acetaldehyde content that is greater than a diacetyl content.

Previously, methods for producing cream flavor in food products included adding diacetyl to the product. However, the preparation and addition of diacetyl to food products can be costly. Other methods of enhancing cream flavor, for example in fermented dairy milk, include modifying the bacterial composition of starter cultures for fermentation. For example, published International Application WO 2012/136832 describes the addition of Lacticaseibacillus rhamnosus, previously classified as Lactobacillus rhamnosus, strain CHCC12697 (accession no. DSM24616) to yogurt starter cultures to increase the level of diacetyl in the fermented dairy milk products. As another example, published International Application WO 2019/081577 describes the use of Lacticaseibacillus rhamnosus strain CHCC15871 (accession no. DSM32666) to increase the level of diacetyl in fermented dairy milk. However, none of these strategies appreciates the increased cream flavor associated, e.g., correlated, with a diacetyl content greater than an acetaldehyde content as demonstrated in the Examples. The methods and compositions provided herein are useful for producing acetaldehyde levels and diacetyl levels that are associated with cream flavor, e.g., increased cream flavor, in thermophilic fermented dairy compositions. The use of bacterial strains described herein for achieving diacetyl and acetaldehyde levels associated with cream flavor as described herein, offers a natural solution to increase cream flavor in thermophilic fermented dairy compositions. The demonstrated ability of the compositions and methods provided herein to increase cream flavor without the use of additives or artificial flavors may be desirable to certain consumers.

In some aspects, the methods and compositions described herein produce a relative content of acetaldehyde to diacetyl correlated with cream flavor by independently modulating (e.g., increasing or decreasing) the levels of acetaldehyde and diacetyl in the thermophilic fermented dairy composition. In some embodiments, the methods and compositions described herein facilitate decreases in the level of acetaldehyde and increases in the level of diacetyl in the thermophilic fermented dairy composition to achieve a relative content of acetaldehyde to diacetyl correlated with cream flavor. In some embodiments, the cream flavor produced by the methods and compositions provided herein is increased compared to methods of producing cream flavor in thermophilic fermented dairy compositions that do not achieve the relative content of acetaldehyde to diacetyl as described herein.

In some embodiments, the methods provided herein include the use of bacteria or bacterial compositions, e.g., starter cultures, including Lacticaseibacillus rhamnosus. In some embodiments, the Lacticaseibacillus rhamnosus is useful for modulating (e.g., independently increasing or decreasing) the acetaldehyde and diacetyl content in a thermophilic fermented dairy composition. In some embodiments, the bacteria and/or bacterial composition, e.g., starter culture, further includes Streptococcus thermophilus. In some embodiments, the bacteria and bacterial compositions described herein, e.g., starter cultures, decrease the level of acetaldehyde in the thermophilic fermented dairy composition. In some embodiments, the bacteria and bacterial compositions described herein, e.g., starter cultures, increase the level of diacetyl in the thermophilic fermented dairy composition. In some embodiments, the bacteria and bacterial compositions described herein, e.g., starter cultures, increase the level of diacetyl or decrease the level of acetaldehyde in the thermophilic fermented dairy composition. In some embodiments, the bacteria and bacterial compositions described herein, e.g., starter cultures, increase the level of diacetyl and decrease the level of acetaldehyde in the thermophilic fermented dairy composition. In some embodiments, the bacteria and bacterial compositions described herein, e.g., starter cultures, increase the level of diacetyl and decrease the level of acetaldehyde in the thermophilic fermented dairy composition such that the diacetyl content in the thermophilic fermented dairy composition is greater than the acetaldehyde content.

In some embodiments, the content of acetaldehyde to diacetyl is expressed as a ratio (e.g., as a quotient) of acetaldehyde content to diacetyl content. In some embodiments, the bacteria and bacterial compositions described herein, e.g., starter cultures, modulate (e.g., independently increase or decrease) the level of acetaldehyde and diacetyl to produce a ratio of acetaldehyde to diacetyl that is less than 1, e.g., less than about 0.9, 0.8, 0.7, 0.6, 0.5, 0.4, 0.3, 0.2, 0.1, 0.05, or 0.01. In some cases, the content of acetaldehyde to diacetyl is expressed as a percentage. For example, in some cases, the bacteria and bacterial compositions described herein, e.g., starter cultures, modulate (e.g., independently increase or decrease) the level of acetaldehyde and diacetyl to produce a percentage of acetaldehyde to diacetyl that is less than 100%, e.g., less than about 90%, 80%, 70%, 60%, 50%, 40%, 30%, 20%, 10%, 5%, or 1%. Any means suitable for numerically expressing the relationship between the acetaldehyde content and the diacetyl content may be used.

In some cases, the cream flavor in the thermophilic fermented dairy composition produced according to the methods and compositions provided herein is not immediately present, e.g., according to an organoleptic profile, in the thermophilic fermented dairy composition following fermentation. For example, in some cases, the acetaldehyde to diacetyl content that is correlated with an increased cream flavor, e.g., a ratio of acetaldehyde to diacetyl that is less than 1, occurs overtime, e.g., an amount of time after fermentation has stopped. As such, in some embodiments, the cream flavor in the thermophilic fermented dairy composition emerges over time as changes in acetaldehyde content and/or diacetyl content evolve. Thus, in some embodiments, the methods provided herein include storing the thermophilic fermented dairy composition, for example as described in Section I-D below. In some embodiments, the thermophilic fermented dairy composition is stored under refrigeration, for example as described in Section I-D below. In some embodiments, the level of acetaldehyde decreases over time in storage. In some embodiments, the level of diacetyl increases over time in storage. In some embodiments, the level of acetaldehyde decreases or the level of diacetyl increases over time in storage. In some embodiments, the level of acetaldehyde decreases and the level of diacetyl increases over time in storage.

In some embodiments, the cream flavor in the thermophilic fermented dairy composition develops during storage. In some embodiments, the cream flavor in the thermophilic fermented dairy composition increases during storage. For example, in some cases, an organoleptic profile indicative of cream flavor changes such that increases in cream aroma and/or cream odor occur with additional time in storage. In some embodiments, the cream flavor in the thermophilic fermented dairy composition develops during storage and then reaches a stable level of cream flavor. For example, in some cases, an organoleptic profile indicative of cream flavor remains stable, e.g., lacks appreciable (e.g., statistically significant, sensorily detectable) increases or decreases in cream aroma and/or cream odor with additional time in storage. Methods for producing and assessing organoleptic profiles include those described in Section I-E and, for example, as described in Example 1.

In some embodiments, a thermophilic fermented dairy composition having cream flavor is a thermophilic fermented dairy composition that has been stored for a duration of time. In some embodiments, the thermophilic fermented dairy composition having cream flavor is a stored thermophilic fermented dairy composition. In some embodiments, the duration of storage of the thermophilic fermented dairy composition is or is about the amount of time needed for the acetaldehyde and diacetyl to decrease and increase, respectively, to levels, e.g., relative quantities, ratios, that correlate with cream flavor in the thermophilic fermented dairy composition, e.g., stored thermophilic fermented dairy composition.

In some embodiments, the cream flavor in the thermophilic fermented dairy composition t produced according to the methods and compositions provided herein is associated, e.g., correlated, with specific levels of diacetyl and acetaldehyde in the thermophilic fermented dairy composition, and relative quantities and ratios thereof. See, e.g., Section I-E below. For example, in some embodiments, the cream flavor is associated with a level of diacetyl that is greater than the level of acetaldehyde. In some embodiments, the cream flavor is associated, e.g., correlated, with a level of acetaldehyde content and a level of diacetyl in the thermophilic fermented dairy composition that results in a ratio of acetaldehyde to diacetyl that is less than 1, e.g., less than about 0.9, 0.85, 0.8, 0.75, 0.7, 0.6, 0.5, 0.4, 0.3, 0.2, 0.1, 0.075, 0.05, 0.025, or 0.01, or is 0. In some embodiments, the level of diacetyl in the thermophilic fermented dairy composition is at least 1 ppm, e.g., at least about 1.5 ppm, 2 ppm, 3 ppm, 4 ppm, 5 ppm, 10 ppm, or 15 ppm. In some embodiments, the level of acetaldehyde in the thermophilic fermented dairy composition is less than 1 ppm, e.g., less than about 0.75 ppm, 0.5 ppm, 0.25 ppm, 0.2 ppm, 0.1 ppm, 0.05 ppm, 0.01 ppm, or below a lower limit of detection and/or quantification by instrumentation. In some embodiments, the lower limit of quantification is 0.125 ppm. In some embodiments, the level of diacetyl content in the thermophilic fermented dairy composition is at least 1 ppm, e.g., at least about 1.5 ppm, 2 ppm, 3 ppm, 4 ppm, 5 ppm, 10 ppm, or 15 ppm, and the level of acetaldehyde is such that the thermophilic fermented dairy composition has a ratio of acetaldehyde to diacetyl that is less than 1, e.g., less than about 0.9, 0.85, 0.8, 0.75, 0.7, 0.6, 0.5, 0.4, 0.3, 0.2, 0.1, 0.075, 0.05, 0.025, or 0.01, or 0 (e.g., when the level of acetaldehyde is below a level of quantification or detection). In some embodiments, the cream flavor of the thermophilic fermented dairy composition is increased compared to a thermophilic fermented dairy composition having an acetaldehyde to diacetyl ratio of, of about, or of greater than 1. The terms level, content, and concentration may be used interchangeably herein to describe amounts of acetaldehyde, diacetyl, or other volatile organic compounds.

In some embodiments, the cream flavor of the thermophilic fermented dairy composition produced according to the methods and compositions provided herein includes or is a cream aroma. Cream aroma is perceived via the retronasal pathway, for example once the product is in the mouth. In some embodiments, the cream flavor of the thermophilic fermented dairy composition produced according to the methods and compositions provided herein includes or is a cream odor. Cream odor is perceived via the nose using the orthonasal pathway (smell). In some embodiments, the cream flavor of the thermophilic fermented dairy composition produced according to the methods and compositions provided herein includes or is a cream aroma or a cream odor. In some embodiments, the cream flavor of the thermophilic fermented dairy composition produced according to the methods and compositions provided herein includes or is a cream aroma and a cream odor.

In some embodiments, the cream flavor of the thermophilic fermented dairy composition produced according to the methods and compositions provided herein is an increased cream flavor. For example, the cream flavor of the thermophilic fermented dairy composition produced according to the methods and compositions provided herein may be increased compared to a thermophilic fermented dairy composition that does not have or is not produced such that levels of acetaldehyde and diacetyl, or relative quantities or ratios, that correlate with the cream flavor according to the compositions and methods provided herein are achieved. Thus, in some embodiments, the cream flavor of the thermophilic fermented dairy composition produced according to the methods and compositions provided herein includes or is an increased cream aroma. In some embodiments, the cream flavor of the thermophilic fermented dairy composition produced according to the methods and compositions provided herein includes or is an increased cream odor. In some embodiments, the cream flavor of the thermophilic fermented dairy composition produced according to the methods and compositions provided herein includes or is an increased cream aroma or an increased cream odor. In some embodiments, the cream flavor of the thermophilic fermented dairy composition produced according to the methods and compositions provided herein includes or is an increased cream aroma and an increased cream odor. In some embodiments, the increase in cream flavor, e.g., increased cream aroma and/or increased cream odor, is determined in comparison to a thermophilic fermented dairy composition that does not have or is not produced such that levels of acetaldehyde and diacetyl, or relative quantities or ratios, that correlate with the cream flavor according to the compositions and methods provided herein are achieved.

In some embodiments, the cream flavor, e.g., increased cream flavor, including cream aromas and/or cream odors thereof, are determined by a trained panel of sensory assessors.

Also provided herein are thermophilic fermented dairy compositions that are food or feed products (e.g., thermophilic fermented dairy products) produced according to the methods provided herein, including embodiments thereof. In some embodiments, the thermophilic fermented dairy composition is a thermophilic fermented dairy product. In some embodiments, the thermophilic fermented dairy product is a yogurt, a cream, a matured cream, a cheese, a fromage frais, a milk beverage, a processed cheese, a cream dessert, a cottage cheese, an infant milk, or a kefir. In some embodiments, the thermophilic fermented dairy product is a stored thermophilic fermented dairy product.

Methods of identifying bacteria capable of producing a thermophilic fermented dairy composition, optionally a stored thermophilic fermented dairy composition, having cream flavor are also provided herein. In some embodiments, the methods of identifying include quantifying acetaldehyde and diacetyl content, and optionally changes thereof, in a thermophilic fermented dairy composition fermented in the presence of one or more bacteria. In some embodiments, the method of identifying includes determining the relative content, e.g., ratio, of acetaldehyde to diacetyl in a thermophilic fermented dairy composition fermented in the presence of one or more bacteria. In some embodiments, the method of identifying includes quantifying acetaldehyde content and diacetyl content, and optionally changes thereof, in a thermophilic fermented dairy composition fermented in the presence of one or more bacteria and stored following fermentation. For example, quantifying acetaldehyde content and diacetyl content, and optionally changes thereof, occurs during storage of the thermophilic fermented dairy composition. In some embodiments, the method of identifying includes determining the relative content, e.g., ratio, of acetaldehyde to diacetyl in a thermophilic fermented dairy composition fermented in the presence of one or more bacteria and stored following fermentation. For example, determining the relative content, e.g., ratio, of acetaldehyde to diacetyl occurs during storage of the thermophilic fermented dairy composition. In some embodiments, the method of identifying includes quantifying acetaldehyde content and diacetyl content, and optionally changes thereof, in a stored thermophilic fermented dairy composition fermented in the presence of one or more bacteria. In some embodiments, the method of identifying includes determining the relative content, e.g., ratio, of acetaldehyde to diacetyl in a stored thermophilic fermented dairy composition fermented in the presence of one or more bacteria. In some embodiments, the quantifying of content and/or determination of relative content, e.g., ratio, as described herein takes place at different time points over the duration of storage. In some embodiments, the quantifying of content and/or determination of relative content, e.g., ratio, as described herein takes place continuously over the duration of storage. In some embodiments, the storage is carried out as described in Section I-D below. In some embodiments, the one or more bacteria useful for achieving diacetyl and/or acetaldehyde contents, and relative quantities and ratios, correlated with cream flavor in a thermophilic fermented dairy milk composition as described herein (see, e.g., Section I-E) are identified as capable of producing a thermophilic fermented dairy composition having cream flavor. In some embodiments, the one or more identified bacteria are contained in a composition such as a starter culture.

The methods and compositions provided herein are useful for naturally producing thermophilic fermented dairy compositions having cream flavor, e.g., an increased cream flavor, without the need for potentially undesirable and/or costly additives. Through the use of bacteria and bacterial compositions for fermentation, as described herein, the methods provided herein circumvent the need for additional processing steps, e.g., introducing additives to the composition, thus allowing production of the thermophilic fermented dairy composition to proceed without added time delays. In addition, the methods and compositions provided herein are useful for producing reduced fat thermophilic fermented dairy compositions having cream flavor, which may increase consumption of reduced fat products and promote nutritional and health benefits. The methods and compositions described herein thus provide numerous advantages while also satisfying a consumer desire for fermented dairy products having a naturally increased cream flavor.

Unless defined otherwise, all technical and scientific terms used herein have the same meaning as commonly understood by one of ordinary skill in the art to which this disclosure belongs.

This disclosure is not limited by the exemplary methods and materials disclosed herein, and any methods and materials similar or equivalent to those described herein can be used in the practice or testing of embodiments of this disclosure.

The headings provided herein are not limitations of the various aspects or embodiments of this disclosure which can be had by reference to the specification as a whole. The section headings used herein are for organizational purposes only and are not to be construed as limiting the subject matter described. Any terms defined are more fully defined by reference to the specification as a whole.

Definitions of terms may appear throughout the specification. It is to be understood that this disclosure is not limited to particular embodiments described, as such may, of course, vary. It is also to be understood that the terminology used herein is for the purpose of describing particular embodiments only and is not intended to be limiting.

It must be noted that as used herein and in the appended claims, the singular forms “a”, “an”, and “the” include plural referents unless the context clearly dictates otherwise. For example, “a” or “an” include “at least one” or “one or more.” The terms “comprising”, “comprises” and “comprised of” as used herein are synonymous with “including”, “includes” or “containing”, “contains”, and grammatical variants thereof, are inclusive or open-ended and do not exclude additional, non-recited members, elements or method steps.

The terms “comprising”, “comprises” and “comprised of”, “including”, “includes” or “containing”, “contains”, and grammatical variants thereof also include the term “consisting of”.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

Values and ranges may be presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context. All values and ranges may implicitly include the term “about” unless the context dictates otherwise.

All publications, including patent documents, scientific articles and databases, referred to in this application are incorporated by reference in their entirety for all purposes to the same extent as if each individual publication were individually incorporated by reference. Nothing herein is to be construed as an admission that such publications constitute prior art. If a definition set forth herein is contrary to or otherwise inconsistent with a definition set forth in the patents, applications, published applications and other publications that are herein incorporated by reference, the definition set forth herein prevails over the definition that is incorporated herein by reference.

### I. METHODS FOR PRODUCING THERMOPHILIC FERMENTED DAIRY COMPOSITIONS HAVING CREAM FLAVOR

The methods and compositions provided herein allow for the production of thermophilic fermented dairy compositions having cream flavor, wherein the cream flavor may be increased compared to thermophilic fermented dairy compositions produced according to different methods. In some embodiments, the cream flavor of the thermophilic fermented dairy composition produced according to the methods provided herein is increased compared to the cream flavor of thermophilic fermented dairy compositions produced according to other methods, for example, that do not control, e.g., modify, the relative amounts of acetaldehyde and diacetyl in the thermophilic fermented dairy composition to levels associated, e.g., correlated, with cream flavor as described herein (see, e.g., Section I-E).

As described in the Examples at Section IV, it was elucidated that thermophilic fermented dairy compositions having increased levels of diacetyl compared to acetaldehyde have increased cream flavor compared to thermophilic fermented dairy composition having about equal amounts of acetaldehyde and diacetyl or where acetaldehyde content is greater than diacetyl content. Thus, the methods and compositions provided herein are designed to achieve a thermophilic fermented dairy composition having relative quantities of acetaldehyde and diacetyl that are associated, e.g., correlated, with increased cream flavor. In some embodiments, the increased cream flavor has an increased cream aroma. In some embodiments, the increased cream flavor has an increased cream odor. In some embodiments, the increased cream flavor has an increased cream aroma or increased cream odor. In some embodiments, the increased cream flavor has an increased cream aroma and increased cream odor.

In some aspects, a method for producing a thermophilic fermented dairy composition having cream flavor is provided, the method including decreasing acetaldehyde content and increasing diacetyl content in a thermophilic fermented dairy composition.

In some aspects, a method for producing a stored thermophilic fermented dairy composition having cream flavor is provided, the method including storing a thermophilic fermented dairy composition to produce the stored thermophilic fermented dairy composition and decreasing acetaldehyde content and increasing diacetyl content in the thermophilic fermented dairy composition during storage.

In some aspects, a method of producing a cream flavor in a thermophilic fermented dairy composition during storage of the thermophilic fermented dairy composition is provided, the method including decreasing acetaldehyde content and increasing diacetyl content in the thermophilic fermented dairy composition during storage.

In some embodiments, the methods provided herein include producing a thermophilic fermented dairy composition. In some embodiments, the method for producing a thermophilic fermented dairy composition includes inoculating a milk substrate, for example as described in Section I-A below, with a bacteria or bacterial composition, for example as described in Section I-B below, and fermenting the inoculated milk substrate, for example as described in Section I-C below.

As indicated above, in some embodiments, the thermophilic fermented dairy composition is stored. A milk substrate that has undergone thermophilic fermentation and is subsequently stored may be referred to herein as a stored thermophilic dairy composition. In some embodiments, the storage is under refrigeration. In some embodiments, the storage of the thermophilic fermented dairy composition, is carried out according to the methods described in Section I-D to produce the stored thermophilic fermented dairy composition.

In some embodiments, the relative quantities of acetaldehyde and diacetyl in the thermophilic fermented dairy composition that associate, e.g., correlate, with cream flavor, e.g., increased cream flavor, are achieved during storage of the thermophilic fermented dairy composition. In some embodiments, the levels, relative quantities, and ratios of acetaldehyde and diacetyl associated with cream flavor are described in Section I-E.

The methods and compositions provided herein are useful for producing a thermophilic fermented dairy composition that is a thermophilic fermented dairy product having cream flavor, e.g., an increased cream flavor. Exemplary thermophilic fermented dairy products are described in Section I-F.

### A. Milk Substrate

In some embodiments, the thermophilic fermented dairy composition having cream flavor is produced by inoculating and thermophilically fermenting a milk substrate. It is contemplated herein that a milk substrate for producing the thermophilic fermented dairy composition may be any raw and/or processed milk material that can be subjected to thermophilic fermentation according to the methods provided herein. Thus, useful milk substrates include, but are not limited to, solutions/suspensions of any milk or milk-like products comprising protein, such as full fat or reduced fat milk, skim milk, buttermilk, reconstituted milk powder, condensed milk, dried milk, whey, whey permeate, whey protein concentrate, or cream. In some embodiments, the milk substrate may originate from any mammal, e.g., being substantially pure mammalian milk, or reconstituted milk powder.

The milk may be in the native state, reconstituted milk, or a skimmed milk. In some embodiments, the milk is supplemented with compounds necessary for the growth of bacteria or for the subsequent processing of fermented milk, such as fat, proteins of a yeast extract, peptone and/or a surfactant, for example.

In some embodiments, milk is a lacteal secretion obtained by milking any mammal, such as cows, sheep, goats, buffaloes, zebras, horses, donkeys, camels, and the like. In some embodiments, the milk substrate is a dairy milk. In some embodiments, the milk is cow milk.

In some embodiments, the milk substrate is a full fat milk. For example, in some cases, the full fat milk is milk that has not had fat, e.g., milk fat, content removed. In some embodiments, the full fat milk is, is about, or is above 3.5% fat by weight. In some embodiments, the full fat milk is or is about 3.25% fat by weight. In some embodiments, the milk substrate is a diluted milk. For example, in some cases, a diluted milk may result in a milk having reduced fat or protein by weight compared to full fat milk. In some embodiments, the milk substrate is a reduced fat milk. For example, in some cases, the reduced fat milk is milk that has had a portion of its fat, e.g., milk fat, content removed. In some embodiments, the portion removed is, is about, or is at least 1%, 5%, 10%, 20%, 30%, 40%, 50%, 60%, 70%, 80%, or 90% of the fat content, e.g., by weight. In some embodiments, the reduced fat milk is or is about 1.6% fat by weight. In some embodiments, the reduced fat milk is or is about 1% fat by weight. In some embodiments, a reduced fat milk may be referred to as a low fat milk. For example, a reduced fat milk that is or is about 1% fat by weight may be referred to as a low fat milk. In some embodiments, the milk substrate is non-fat milk, also referred to as skim milk. For example, in some cases, the non-fat milk is milk that has had about 100% of its fat, e.g., milk fat, content removed. In some embodiments, the non-fat milk is, is about, or is less than about 0.3% fat by weight. In some embodiments, the non-fat milk is or is about 0% fat by weight. In some embodiments, the percentage of fat by weight in a non-fat milk is negligible, e.g., at or below a level of detection or quantification. Suitable methods for measuring fat in milk, e.g., milk fat, include, but are not limited to, the Röse-Gottlieb reference method, the Gerber butyrometric method, the Mojonnier method, spectrophotometry, e.g., UV, and spectroscopy, e.g., infrared. In some embodiment, the full fat, reduced fat, low fat, and/or non-fat milk referred to herein is a dairy milk, optionally cow milk.

The protein content may not be essential to the present invention. Protein content of the milk substrate used according to the present invention may be from e.g. 1% to about 20%, such as in fermented milk enriched or concentrated with respect to protein content. However, in some embodiments, the milk substrate has a reduced protein content. In some embodiments, the milk substrate has a protein content of or of about 3%. In some embodiments, the milk substrate has a protein content of or of about 3.4%. In some embodiments, the milk substrate is commercial UHT milk. In some embodiments, the milk substrate is a fresh milk. In some embodiments, the milk substrate is a fresh commercial milk. In some embodiments, the milk substrate, such as a fresh milk, has undergone thermization. In some embodiments, the milk substrate, such as a fresh milk, has undergone pasteurization. In some embodiments, the milk substrate, such as a fresh milk, has undergone thermization and pasteurization.

In some embodiments, the milk substrate has low, undetectable, or unquantifiable levels of acetaldehyde. In some embodiments, the milk substrate has low, undetectable, or unquantifiable levels of diacetyl. In some embodiments, the milk substrate has low, undetectable, or unquantifiable levels of acetaldehyde and diacetyl. Methods for detecting and/or quantifying levels of volatile organic compounds, such as acetaldehyde and diacetyl, include but are not limited to mass spectrometry, e.g., gas chromatograph-mass spectrometry (GC-MS), selected ion flow tube mass spectrometry (SIFT-MS), proton transfer reaction mass spectrometry (PTR-MS), ion-molecule reaction mass spectrometry (IMR-MS). In some embodiments, low levels of acetaldehyde or diacetyl are less than 0.5 ppm, e.g., less than 0.4, 0.3, 0.2, or 0.1 ppm. In some embodiments, low levels of acetaldehyde or diacetyl are between about 0.5 to about 0.1 ppm, about 0.4 to about 0.1 ppm, about 0.3 to about 0.1 ppm, or about 0.2 to about 0.1 ppm. In some embodiments, undetectable levels of acetaldehyde and diacetyl refer to amounts of the volatile organic compound that are below the lower limit for detection and/or quantification by the instrument used to detect the compound. In some embodiments, acetaldehyde and diacetyl are detected and quantified using GC-MS. In some embodiments, the lower limit for quantification is 0.125 ppm. In some embodiments, thermophilic fermentation of the milk substrate according to the methods and compositions described herein results in an increase in an amount of acetaldehyde and/or diacetyl compared to the amount of acetaldehyde and/or diacetyl in the milk substrate prior to fermentation.

In some cases, the milk substrate may be homogenized and/or treated with heat, i.e., pasteurized. In some embodiments, the milk substrate is thermophilically fermented. Prior to thermophilic fermentation, the milk substrate may be homogenized, pasteurized, and/or undergo thermization according to methods known in the art.

In some cases, homogenization is or includes intensive mixing to obtain a soluble suspension or emulsion. If homogenization is performed prior to thermophilic fermentation, it may be performed to break up fat in the milk substrate, e.g., milk fat, into smaller sizes so that it no longer separates from the milk. This may be accomplished by forcing the milk at high pressure through small orifices.

In some embodiments, pasteurizing includes treatment of the milk substrate to reduce or eliminate the presence of live organisms, such as microorganisms. In some embodiments, pasteurization is attained by maintaining a specified temperature for a specified period of time. In some embodiments, the specific temperature is attained by heating. The temperature and duration may be selected in order to kill or inactivate certain bacteria, such as harmful bacteria. In some embodiments, the milk substrate is heated to a temperature between or between about 80 to about 120° C., inclusive. In some embodiments, the milk substrate is heated to a temperature between or between about 85 to about 100° C., inclusive. In some embodiments, the milk substrate is heated to a temperature between or between about 85 to about 95° C., inclusive. In some embodiments, the milk substrate is heated to a temperature of or of about 90° C., e.g., 90° C. +/−0.2° C. In some embodiments, the milk substrate is heated for or for about 5 to 20 minutes. In some embodiments, the milk substrate is heated for or for about 5 to 15 minutes. In some embodiments, the milk substrate is heated for or for about 10 minutes, e.g., 10 min±1 min.

In some embodiments, the milk substrate may undergo thermization. In some cases, thermization is useful for preserving the properties of the milk substrate while significantly reducing, eliminating, or inactivating unwanted or harmful bacteria. In some embodiments, thermization includes heating the milk substrate to a temperature of or of about 57° C. In some embodiments, thermization includes heating the milk substrate to a temperature in the range of or of about 64 to 69° C. In some embodiments, the milk substrate is heated for at least 15 seconds. For example, when the milk substrate is heated to a temperature of or of about 57° C., the duration of heating is for at least 15 seconds. In some embodiments, the milk substrate is heated for or for about 5 to 40 seconds. For example, when the milk substrate is heated to a temperature of or of about 64 to 69° C., the duration of heating is or is about 5 to 40 seconds. Thermization can be employed depending on milk quality and/or fermentation technology to be used.

In some embodiments, e.g., after heat treating (e.g., pasteurization, thermization), the milk substrate is cooled. In some embodiments, the milk substrate is cooled to a temperature between or between about 30 to about 50° C., inclusive. In some embodiments, the milk substrate is cooled to a temperature between or between about 35 to about 45° C., inclusive. In some embodiments, the milk substrate is cooled to a temperature of or of about 43° C. In some embodiments, the cooling is rapid cooling. In some embodiments, the milk substrate is cooled for less than 20, 15, 10, 9, 8, 7, 6, 5, 4, 3, 2, or 1 minute. In some embodiments, the milk substrate is cooled for less than 10 minutes. In some embodiments, the milk substrate is cooled for less than 5 minutes. In some embodiments, the milk substrate is cooled for or for about 10 minutes. In some embodiments, the milk substrate is cooled for or for about 5 minutes. In some embodiments, the milk substrate is cooled to the temperature at which thermophilic fermentation will proceed.

In some embodiments, the milk substrate is inoculated with a bacterial composition, e.g., starter culture as described in Section I-B, to facilitate thermophilic fermentation.

### B. Bacteria and Bacterial Compositions

The milk substrate, e.g., as described in Section I-A, may be inoculated with one or more bacterial strains, e.g., a starter culture, to facilitate thermophilic fermentation. In some embodiments, the one or more bacteria are contained in a bacterial composition, such as a starter culture. In some embodiments, the one or more bacterial strains are contained in a starter culture. In some embodiments, the starter culture is a preparation of living bacteria able to assist in fermentation, e.g., thermophilic fermentation. In some embodiments, the milk substrate is inoculated with a starter culture. In some embodiments, the milk substrate is inoculated with a starter culture and a protective culture. In some embodiments, a protective culture is a culture able to reduce or prevent the growth of biological contaminants, such as yeast and mold. In some embodiments, the protective culture may include one or more bacterial strains described herein, e.g., an L. rhamnosus strain described herein. The terms inoculating and adding may be used interchangeably to refer to contacting a milk substrate with with one or more bacteria, for example as contained in a bacterial composition, e.g., starter culture, protective culture. In some embodiments, the milk substrate is inoculated with one or more bacterial strains separately. For example, the strains are not mixed together prior to being added to the milk substrate. In some embodiments, the strains are mixed together prior to being added to the milk substrate. Regardless of how a strain is added to the milk substrate, the strain or mixtures of strains used for inoculation may be referred to as or included in a starter culture or protective culture. In some embodiments, the starter culture is a thermophilic starter culture, and the composition of the starter culture is suitable for thermophilic fermentation as described in Section I-C.

In some embodiments, thermophilic microorganisms, such as thermophilic bacteria, refer to microorganisms that function preferentially at temperatures above 37° C., e.g., 37 to 50° C.

In some embodiments, the starter culture is a pure culture, i.e., comprises or consists of a single bacterial strain. In some embodiments, the starter culture is a mixed culture, i.e. comprises or consists of at least one bacterial strain of the invention as described herein and at least one other bacterial strain. For example, at least 1 or more, and in particular 1, 2, 3, 4 or 5, other bacterial strains are included in the starter culture.

In some embodiments, the starter culture contains one or more lactic acid bacteria. As it is normal in lactic acid bacterial fermentation processes to apply a mixed culture as a starter culture, the composition may in some embodiments include a multiplicity of strains either belonging to the same species or belonging to different species. For example, in some cases, the lactic acid bacteria in the starter culture is or includes a mixture of a Lactobacillus delbrueckii subsp bulgaricus strain, a Streptococcus thermophilus strain, and an L. rhamnosus strain.

In some embodiments, the starter culture includes at least bacteria from the genera Streptococcus and Lacticaseibacillus. In some embodiments, the starter culture includes or consists of bacteria from the genus Lactococcus, Lactobacillus, Streptococcus, Lacticaseibacillus, Leuconostoc, Pediococcus, Enterococcus, Bifidobacterium, Paralactobacillus, Acetilactobacillus, Agrilactobacillus, Amylolactobacillus, Apilactobacillus, Bombilactobacillus, Companilactobacillus, Dellaglioa, Fructilactobacillus, Furfurilactobacillus, Holzapfelia, Lacticaseibacillus, Lactiplantibacillus, Lapidilactobacillus, Latilactobacillus, Lentilactobacillus, Levilactobacillus, Ligilactobacillus, Limosilactobacillus, Liquorilactobacillus, Loigolactobacilus, Paucilactobacillus, Schleiferilactobacillus, Secundilactobacillus, or any combination thereof. In some embodiments, the starter culture includes or consists of bacteria from the genus Lactococcus, Lactobacillus, Streptococcus, Lacticaseibacillus, Leuconostoc, Pediococcus, Enterococcus or Bifidobacterium, or any combination thereof. In some embodiments, the starter culture includes or consists of bacteria from the genus Lactococcus, Lactobacillus, Streptococcus, Lacticaseibacillus, Leuconostoc, Pediococcus, or Bifidobacterium, or any combination thereof. In some embodiments, the starter culture includes or consists of bacteria from the genus Lactococcus, Lactobacillus, Streptococcus, Lacticaseibacillus, or any combination thereof. In some embodiments, the starter culture includes or consists of bacteria from the genus Lactobacillus, Streptococcus, Lacticaseibacillus, or any combination thereof. In some embodiments, the starter culture includes or consists of bacteria from the genus Streptococcus and Lacticaseibacillus.

In some embodiments, the starter culture includes at least a Streptococcus thermophilus strain and a Lacticaseibacillus rhamnosus strain. In some embodiments, the starter culture includes one or more of a Streptococcus thermophilus strain, a Lactobacillus acidophilus strain, a Lacticaseibacillus rhamnosus strain, a Bifidobacterium lactis strain, a Limosilactobacillus fermentum strain, a Lacticaseibacillus paracasei strain, a Lactiplantibacillus plantarum strain, a Lactobacillus delbrueckii subsp bulgaricus strain, a Propionibacteria freudenreichii strain, a Pediococcus acidilactici strain, an Enterococcus faecium strain, a Lactococcus lactis strain, a Lactococcus cremoris strain or any combination of the foregoing. In some embodiments, the starter culture includes one or more of a Streptococcus thermophilus strain, a Lactobacillus acidophilus strain, a Lacticaseibacillus rhamnosus strain, a Bifidobacterium lactis strain, a Limosilactobacillus fermentum strain, a Lacticaseibacillus paracasei strain, a Lactiplantibacillus plantarum strain, a Lactobacillus delbrueckii subsp bulgaricus strain, a Propionibacteria freudenreichii strain, a Pediococcus acidilactici strain, a Lactococcus lactis strain, Lactococcus cremoris strain, or any combination of the foregoing. In some embodiments, the starter culture includes one or more of a Streptococcus thermophilus strain, a Lacticaseibacillus rhamnosus strain, a Lactobacillus delbrueckii subsp bulgaricus strain, or any combination of the foregoing. In some embodiments, the starter culture includes a Streptococcus thermophilus strain and a Lacticaseibacillus rhamnosus strain. In some embodiments, the starter culture includes more than one strain of a species.

In some embodiments, the starter culture includes at least one Lactococcus strain. For example, the starter culture may include one or more of a Lactococcus strain known in the art, such as a strain of Lactococcus cremoris subsp cremoris (previously referred to as Lactococcus lactis subsp. cremoris), Lactococcus lactis subsp hordniae, or Lactococcus lactis subsp lactis. In some embodiments, the starter culture includes a Lactococcus cremoris subsp cremoris and/or a Lactococcus lactis subsp lactis strain.

As described in the Examples, thermophilic fermented dairy compositions inoculated and fermented with starter cultures including Streptococcus and Lacticaseibacillus strains had a diacetyl content greater than a content of acetaldehyde. Thermophilic fermented dairy compositions with higher levels of diacetyl than acetaldehyde were found to correlate with an increased cream flavor, as determined by a trained panel of sensory assessors, compared to thermophilic fermented dairy compositions with similar amounts of acetaldehyde and diacetyl or with an acetaldehyde content greater than the diacetyl content. Thus, in some embodiments, the starter culture is or includes strains of the species Streptococcus thermophilus and Lacticaseibacillus rhamnosus.

In some embodiments, the starter culture includes one or more Lacticaseibacillus rhamnosus strains. In some embodiments, the starter culture includes Lacticaseibacillus rhamnosus strain DGCC1179 that was deposited with the German Collection of Microorganisms and Cell Cultures (DSMZ) under accession no. DSM33650 or a mutant strain thereof. In some embodiments, the mutant strain is obtained by using the deposited strain as starting material. In some embodiments, the mutant strain is a strain having all of the identifying characteristics of the strain deposited at the DSMZ under number DSM33650. In some embodiments, the starter culture includes Lacticaseibacillus rhamnosus strain DGCC1460 that was deposited at the DSMZ under accession no. DSM22876 or a mutant strain thereof. In some embodiments, the mutant strain is obtained by using the deposited strain as starting material. In some embodiments, the mutant strain is a strain having all of the identifying characteristics of the strain deposited at DSMZ under number DSM22876. In some embodiments, the starter culture includes Lacticaseibacillus rhamnosus strain DGCC9913 that was deposited at the DSMZ under accession no. DSM22193 or a mutant strain thereof. In some embodiments, the mutant strain is obtained by using the deposited strain as starting material. In some embodiments, the mutant strain is a strain having all of the identifying characteristics of the strain deposited at DSMZ under number DSM22193.

In some embodiments, the starter culture includes one or more Streptococcus thermophilus strains. In some embodiments, the starter culture includes Streptococcus thermophilus strain DGCC11042 that was deposited at the DSMZ under accession no. DSM33651 or a mutant strain thereof. In some embodiments, the mutant strain is obtained by using the deposited strain as starting material. In some embodiments, the mutant strain is a strain having all of the identifying characteristics of the strain deposited at DSMZ under number DSM33651. In some embodiments, the starter culture includes Streptococcus thermophilus strain DGCC12456 that was deposited at the DSMZ under accession no. DSM33849 or a mutant strain thereof. In some embodiments, the mutant strain is obtained by using the deposited strain as starting material. In some embodiments, the mutant strain is a strain having all of the identifying characteristics of the strain deposited at DSMZ under number DSM33849. In some embodiments, the starter culture includes Streptococcus thermophilus strain DGCC7693 that was deposited at the DSMZ under accession no. DSM33829 or a mutant strain thereof. In some embodiments, the mutant strain is obtained by using the deposited strain as starting material. In some embodiments, the mutant strain is a strain having all of the identifying characteristics of the strain deposited at DSMZ under number DSM33829.

In some embodiments, the starter culture includes one or more strains of Lacticaseibacillus rhamnosus and one or more strains of Streptococcus thermophilus.

In some embodiments, the starter culture includes or consists of strain DSM33650 or a mutant thereof and strain DSM33651 or a mutant thereof. In some embodiments, the starter culture includes or consists of strain DSM33650 or a mutant thereof and strain DSM33849 or a mutant thereof. In some embodiments, the starter culture includes or consists of strain DSM33650 or a mutant thereof and strain DSM33829 or a mutant thereof. In some embodiments, the starter culture includes or consists of strain DSM22876 or a mutant thereof and strain DSM33651 or a mutant thereof. In some embodiments, the starter culture includes or consists of strain DSM22876 or a mutant thereof and strain DSM33849 or a mutant thereof. In some embodiments, the starter culture includes or consists of strain DSM22876 or a mutant thereof and strain DSM33829 or a mutant thereof. In some embodiments, the starter culture includes or consists of strain DSM22193 or a mutant thereof and strain DSM33651 or a mutant thereof. In some embodiments, the starter culture includes or consists of strain DSM22193 or a mutant thereof and strain DSM33849 or a mutant thereof. In some embodiments, the starter culture includes or consists of strain DSM22193 or a mutant thereof and strain DSM33829 or a mutant thereof. In some embodiments, the mutant strain is obtained by using the deposited strain as starting material. In some embodiments, the mutant is a strain having all of the identifying characteristics of the strain deposited at DSMZ.

As used herein, a mutant may be understood as a strain derived from a strain described herein by means of e.g., genetic engineering, radiation, UV light, and/or chemical treatment and/or methods that induce changes in the genome. The mutant may be a functionally equivalent mutant, e.g., a mutant that has substantially the same or improved properties (e.g., regarding diacetyl and acetaldehyde content regulation and/or production in thermophilic fermented dairy compositions; flavor, e.g., cream flavor; post-acidification; acidification speed; and/or phage robustness) as the parent strain. In some embodiments, the mutant decreases acetaldehyde content in thermophilic fermented dairy compositions. In some embodiments, the mutant increases diacetyl content in thermophilic fermented dairy compositions. In some embodiments, the mutant decreases acetaldehyde and/or increases diacetyl content in thermophilic fermented dairy compositions. In some embodiments, the mutant decreases acetaldehyde and increases diacetyl content in thermophilic fermented dairy compositions when used in combination with other strains or mutants. In some embodiments, the mutant either alone or in combination with another strain or mutant is useful for achieving levels of acetaldehyde and diacetyl, and relative quantities and ratios thereof, that correlate with a cream flavor, e.g., increased cream flavor, in thermophilic fermented dairy compositions. In some embodiments, the mutant may demonstrate phage resistance and/or reduced phage sensitivity that is the same or different from the phage resistance and/or reduced phage sensitivity of the parent strain. In some embodiments, the phage resistance and/or reduced phage sensitivity may result from differences at one or more CRISPR loci compared to the parent strain. In some embodiments, the difference may be the addition of one or more spacers in one or more CRISPR loci. In some embodiments, the phage resistance and/or reduced phage sensitivity may result from non-CRISPR-mediated phage resistance mechanisms that differ from those of the parent strain. Thus, in some embodiments, the CRISPR loci of the mutant strain are identical to the CRISPR loci of the parent strain but the mutant strain displays different phage resistances and/or reduced phage sensitivities compared to the parent strain. Methods for inducing or generating phage resistance or reduced sensitivity and detecting such features are known in the art. Non-limiting methods for inducing phage resistance or reduced phage sensitivity may be found, for example, in published international applications WO 2007/025097, WO 2007/136815, and WO 2008/108989, which are incorporated by reference in their entirety. Such mutants are a part of the present invention.

A mutant, e.g., mutant strain, as referred to herein may be a strain obtained by subjecting a strain described herein to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenization treatments (a single treatment should be understood as containing one mutagenization step followed by a screening/selection step). In some embodiments, no more than 20, or no more than 10, or no more than 5 treatments (or screening/selection steps) are carried out to produce a mutant. In some embodiments, less than 5%, or less than 1% or less than 0.1% of the nucleotides in the bacterial genome have been shifted with another nucleotide, deleted, substituted or added compared to the mother strain. In some embodiments, the mutant contains no more than 20, in particular no more than 10, in particular no more than 5, in particular no more than 4, in particular no more than 3, in particular no more than 2, and in particular no more than 1 nucleotide mutation of the bacterial genome as compared to the mother strain, wherein a mutation is a substitution, an insertion or a deletion of a nucleotide. In some embodiments, a mutant is obtained by subjecting a strain described herein to a selective pressure or a stressor.

In some embodiments, the starter culture, either as a pure or mixed culture as defined above, is in frozen, dried, freeze-dried, liquid or solid format, in the form of pellets or frozen pellets, or in a powder or dried powder. In some embodiments, the starter culture is in a frozen format or in the form of pellets or frozen pellets, in particular contained into one or more box or sachet. In some embodiments, the starter cultures as defined herein are in a powder form, such as a dried or freeze-dried powder, in particular contained into one or more box or sachet. In some embodiments, the liquid format is a bulk starter, such as a bacterial strain or mixture of bacterial strains previously propagated in a growth medium to obtain the required concentration for inoculation.

Commercial non-concentrated cultures of bacteria are referred to in industry as ‘mother cultures’, and are propagated at the production site, for example a dairy, before being added to an edible starting material, such as milk substrate, for fermentation.

In some embodiments, the starter culture, either as a pure culture or mixed culture as defined above, and whatever the format (frozen, dried, freeze-dried, liquid or solid format, in the form of pellets or frozen pellets, or in a powder or dried powder) includes the bacterial strain(s) described herein in a concentration in the range of or of about 10⁵ to 10¹³ cfu (colony forming units) per gram of the starter culture. In some embodiments, the concentration of the bacterial strain(s) described herein within the starter culture of the invention is in the range of or of about 10⁵ to 10¹³ cfu per gram of the starter culture, and in particular at least 10⁶, at least 10⁷, at least 10⁸, at least 10⁹, at least 10¹⁰ or at least 10¹¹ CFU/g of the starter culture. In some embodiments, when in the form of frozen or dried concentrate, the concentration of bacterial strain(s) described herein—as pure culture or as a mixed culture—within the starter culture is in the range of or of about 10⁶ to 10¹³ cfu/g of frozen concentrate or dried concentrate, and more preferably at least 10⁷, at least 10⁸, at least 10⁹, at least 10¹⁰, at least 10¹¹, at least 10¹², or at least 10¹³ cfu/g of frozen concentrate or dried concentrate. In some embodiments, the concentration of the bacterial strain(s) described herein within the starter culture of the invention is the range of or of about 10⁵ to 10⁹ CFU/g of the starter culture. In some embodiments, the concentration of the bacterial strain(s) described herein within the starter culture of the invention is the range of or of about 10⁶ to 10⁹ CFU/g of the starter culture. In some embodiments, the concentration of the bacterial strain(s) described herein within the starter culture of the invention is the range of or of about 10⁶ to 10⁸ CFU/g of the starter culture. In some embodiments, the concentration of the bacterial strain(s) described herein within the starter culture of the invention is the range of or of about 10⁶ to 10⁷ CFU/g of the starter culture. In some embodiments, the concentration of the bacterial strain(s) described herein within the starter culture of the invention is, is at least, or is about 10⁶ CFU/g of the starter culture. In some embodiments, the concentration of the bacterial strain(s) described herein within the starter culture of the invention is, is at least, or is about 10⁷ CFU/g of the starter culture.

Starter cultures may be prepared by techniques well known in the art such as those disclosed in U.S. Pat. No. 4,621,058. By way of example, starter cultures may be prepared by the introduction of an inoculum, for example a bacterium, to a growth medium to produce an inoculated medium and ripening the inoculated medium to produce a starter culture. Dried starter cultures may be prepared by techniques well known in the art, such as those discussed in U.S. Pat. Nos. 4,423,079 and 4,140,800. In some embodiments, the addition of the starter culture to the milk substrate is direct, e.g., as a direct vat inoculate, direct vat starter, direct vat set culture.

In some embodiments, the milk substrate may be inoculated with the starter culture prior to initiating thermophilic fermentation. In some embodiments, the milk substrate is inoculated with the starter culture following, e.g., at a time point after, thermophilic fermentation is initiated. In some embodiments, the milk substrate is inoculated with the starter culture during thermophilic fermentation. For example, in some cases where strains of the starter culture are added separately, e.g., not in a mixture, the strains may be added at different times before or during fermentation.

Further microorganisms and/or additives may be added to the milk substrate before, during or after fermentation, e.g., thermophilic fermentation, of the milk substrate. For example, boosters, such as yeast extracts and/or amino acid containing compositions may be added to the milk substrate in addition to a starter culture, to support or promote microorganism function and facilitate successful production of the thermophilic fermented dairy composition. Microorganisms that may be added to the milk substrate include those that will contribute in an advantageous manner to the properties of the thermophilic fermented dairy composition. For example, added microorganisms, yeast extracts, or amino acid compositions may improve or support diacetyl production, acetaldehyde conversion and breakdown, the viscosity, gel stiffness, mouth coating, flavor, post acidification, and/or acidification speed in the thermophilic fermented dairy composition. Optionally, other ingredients may be added to the milk substrate, such as colors, stabilizers, e.g., pectin, starch, modified starch, CMC, etc.; or polyunsaturated fatty acids, e.g. omega-3 fatty acids. Such ingredients may be added at any point during the production process, e.g. before or after fermentation.

Components such as cryoprotectants and/or conventional additives including nutrients such as yeast extracts, sugars and vitamins, e.g., vitamin A, C, D, K or vitamins of the vitamin B family may also be added to the milk substrate either with or separate from the start culture or other additives. Suitable cryoprotectants that may be added include components that improve the cold tolerance of the microorganisms, such as mannitol, sorbitol, sodium tripolyphosphate, xylitol, glycerol, raffinose, maltodextrin, erythritol, threitol, trehalose, glucose and fructose. Other additives may include carbohydrates, flavors, minerals, enzymes (e.g., rennet, lactase and/or phospholipase).

In some embodiments, additives, microorganisms, or other components as described in the preceding paragraphs are added simultaneously or nearly simultaneously with the starter culture to the milk substrate, e.g., at the same time as inoculation of the milk substrate with the starter culture. In some embodiments, additives, microorganisms, or other components as described in the preceding paragraphs are added to the milk substrate before, e.g., temporally prior to, inoculation of the milk substrate with the starter culture. In some embodiments, additives, microorganisms, or other components as described in the preceding paragraphs are added after, e.g., temporally following, inoculation of the milk substrate with the starter culture. In some embodiments, additives, microorganisms, or other components as described in the preceding paragraphs may be added to the milk substrate before, during, or after fermentation, for example independent of the inoculation with the starter culture.

In some embodiments, the milk substrate is inoculated with the starter culture, and optionally additives, microorganisms, or other components as described in the preceding paragraphs above, by any suitable method. For example, the milk substrate may be inoculated by direct inoculation into a fermentation vessel.

### C. Thermophilic Fermentation

The methods for producing a thermophilic fermented dairy composition having cream flavor provided herein include thermophilic fermentation of a milk substrate, for example as described in Section I-A, inoculated with a starter culture, and optionally other additives, microorganisms, and components, as described in Section I-B.

Fermentation according to the methods provided herein facilitates the conversion of carbohydrates into alcohols or acids through the action of a microorganism, e.g., bacteria present, for example, in a starter culture. In some embodiments, fermentation according to the methods provided herein includes conversion of lactose to lactic acid.

Thermophilic fermentation processes may be selected to optimize parameters such as temperature, oxygen, process time to achieve a thermophilic fermented dairy composition having cream flavor as described herein.

In some embodiments, the inoculated milk substrate is fermented at a temperature at or above about 33° C., 35° C., 36° C., 37° C., 38° C., 39° C., 40° C., 41° C., 42° C., 43° C., 44° C., 45° C., or 50° C. In some embodiments, the inoculated milk substrate is fermented at a temperature of between or between about 35 to 47° C., 35 to 46° C., 35 to 45° C., 35 to 44° C., 35 to 43° C., 36 to 43° C., 37 to 43° C., 38 to 43° C., 39 to 43° C., 40 to 43° C., 41 to 43° C., or 42 to 43° C. In some embodiments, the inoculated milk substrate is fermented at a temperature of or of about 40° C., 41° C., 42° C., 43° C., or 44° C. In some embodiments, the inoculated milk substrate is fermented at a temperature of or of about 40° C., e.g., 40±1° C. In some embodiments, the inoculated milk substrate is fermented at a temperature of or of about 41° C., e.g., 41±1° C. In some embodiments, the inoculated milk substrate is fermented at a temperature of or of about 42° C., e.g., 42±1° C. In some embodiments, the inoculated milk substrate is fermented at a temperature of or of about 43° C., e.g., 43±1° C. In some embodiments, the inoculated milk substrate is fermented at a temperature of or of about 44° C., e.g., 44±1° C.

In some embodiments, thermophilic fermentation of the inoculated milk substrate is carried out for at least 4 hours, e.g., 5, 6, 7, 8, 9, 10, 15, 20, or 24 hours. In some embodiments, thermophilic fermentation of the inoculated milk substrate is carried out for or for about 4, 5, 6, 7, 8, 9, 10, 11, 12, 13, 14, 15, 16, 17, 18, 19, 20, 21, 22, 23, or 24 hours. In some embodiments, thermophilic fermentation of the inoculated milk substrate is carried out for at most 24 hours. In some embodiments, thermophilic fermentation of the inoculated milk substrate is carried out for between or between about 4 to 24, 4 to 23, 4 to 22, 4 to 21, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 4 to 7, 4 to 6, or 4 to 5 hours. In some embodiments, thermophilic fermentation of the inoculated milk substrate is carried out for or for about 15 hours. In some embodiments, thermophilic fermentation of the inoculated milk substrate is carried out for or for about 14 hours. In some embodiments, thermophilic fermentation of the inoculated milk substrate is carried out for or for about 13 hours. In some embodiments, thermophilic fermentation of the inoculated milk substrate is carried out for or for about 12 hours. In some embodiments, thermophilic fermentation of the inoculated milk substrate is carried out for or for about 11 hours. In some embodiments, thermophilic fermentation of the inoculated milk substrate is carried out for or for about 10 hours. In some embodiments, thermophilic fermentation of the inoculated milk substrate is carried out for or for about 9 hours. In some embodiments, thermophilic fermentation of the inoculated milk substrate is carried out for or for about 8 hours. In some embodiments, thermophilic fermentation of the inoculated milk substrate is carried out for or for about 7 hours. In some embodiments, thermophilic fermentation of the inoculated milk substrate is carried out for or for about 6 hours. In some embodiments, thermophilic fermentation of the inoculated milk substrate is carried out for or for about 5 hours. In some embodiments, thermophilic fermentation of the inoculated milk substrate is carried out for or for about 4 hours.

In some embodiments, thermophilic fermentation of the inoculated milk substrate is carried out for an amount of time needed for the milk substrate to reach a target pH. In some embodiments, the target pH is a suitable pH for producing the thermophilic fermented dairy composition, e.g., as described in Section I-E below. In some embodiments, the target pH is in a range of or of about 3.8 to 5. In some embodiments, the target pH is in a range of or of about 4 to 5. In some embodiments, the target pH is or is about 4.1, 4.2, 4.3, 4.4., 4.5, 4.6, or 4.7. In some embodiments, the target pH is in a range of or of about 4.2 to 4.7. In some embodiments, the target pH is or is about 4.6.

After thermophilic fermentation, in some embodiments, the thermophilic fermented dairy composition has a pH in the range of or of about 3.8 to 5. In some embodiments, the thermophilic fermented dairy composition has a pH in the range of or of about 4 to 5. In some embodiments, the thermophilic fermented dairy composition has a pH of or of about 4.1, 4.2, 4.3, 4.4., 4.5, 4.6, or 4.7. In some embodiments, the thermophilic fermented dairy composition has a pH in the range of or of about 4.2 to 4.7. In some embodiments, the target pH is or is about 4.6.

In some embodiments, the pH of the inoculated milk substrate is measured, e.g., monitored, during thermophilic fermentation. In some embodiments, the pH of the inoculated milk substrate is measured, e.g., monitored, at specific time points during thermophilic fermentation. For example, a pH measurement may be taken every 5, 10, 15, 20, 25, 30, 35, 40, 45, 50, 55, or 60 minutes, or every hour, 2 hours, 3 hours, 4 hours, or 5 hours. In some embodiments, the pH of the inoculated milk substrate is measured, e.g., monitored, continuously during thermophilic fermentation.

In some embodiments, the thermophilic fermented dairy composition, produced by thermophilic fermentation of a milk substrate with a starter culture as described herein, contains levels of acetaldehyde and/or diacetyl that are increased compared to the levels of acetaldehyde and/or diacetyl in the milk substrate prior to thermophilic fermentation.

### D. Storage

In some aspects, the thermophilic fermented dairy composition produced according to the methods provided herein is stored. For example, in some embodiments, following thermophilic fermentation of the inoculated substrate as described herein (see, Sections I-A, I-B, and I-C), the thermophilic fermented dairy composition is stored to produce a stored thermophilic fermented dairy composition.

In some embodiments, the thermophilic fermented dairy composition, e.g., stored thermophilic fermented dairy composition, is stored in one or more containers. Suitable containers for storage included, but are not limited to, containers capable of securing and protecting the thermophilic fermented dairy composition, e.g., protecting the thermophilic fermented dairy composition from contamination. In some embodiments, the thermophilic fermented dairy composition is packaged in a sealed container. In some embodiments, the thermophilic fermented dairy composition is packaged in a container after thermophilic fermentation has occurred. In some embodiments, the thermophilic fermentation occurs in a container that can be sealed. For example, in some cases, a substrate, e.g., inoculated substrate, may be fermented in a container such that at the end of fermentation the container contains a thermophilic fermented dairy composition and the container is then sealed. In some embodiments, the vegetal base is inoculated prior to placement in the container for fermentation. In some embodiments, the vegetal base is inoculated after to placement in the container for fermentation.

In some embodiments, the one or more containers are useful for storing the thermophilic fermented dairy composition at a cool temperature, e.g., a cooler temperature than used for thermophilic fermentation, such as at or at about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C. or 26° C. In some embodiments, the thermophilic fermented dairy composition is stored at a temperature in the range of or of about 1 to 26° C., 1 to 24° C., 1 to 22° C., 1 to 20° C., 1 to 18° C., 1 to 16° C., 1 to 14° C., 1 to 12° C., 1 to 10° C., 1 to 8° C., 1 to 6° C., 1 to 4° C., or 1 to 2° C. In some embodiments, the thermophilic fermented dairy composition is stored at a temperature in the range of or of about 1 to 14° C., 1 to 12° C., 1 to 10° C., 1 to 8° C., 1 to 6° C., or 1 to 4° C. In some embodiments, the thermophilic fermented dairy composition is stored at a temperature in the range of or of about 4 to 8° C. In some embodiments, the thermophilic fermented dairy composition is stored at or at about a temperature of 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C. or 26° C. In some embodiments, the thermophilic fermented dairy composition is stored at or at about a temperature of 1° C., 2° C., 3° C., 4° C., 5° C., 6° C., 7° C., 8° C., 9° C., 10° C., 11° C., 12° C., 13° C. or 14° C. In some embodiments, the thermophilic fermented dairy composition is stored at or at about a temperature of 2° C., 3° C., 4° C., 5° C., 6° C., 7° C., or 8° C. In some embodiments, the thermophilic fermented dairy composition is stored at or at about a temperature of 6° C., e.g., 6±1° C.

In some embodiments, stored thermophilic fermented dairy composition maintains a target pH achieved during thermophilic fermentation. In some embodiments, the target pH is a suitable pH for a thermophilic fermented dairy composition, e.g., as described in Section I-F below. In some embodiments, the target pH is in a range of or of about 3.8 to 5. In some embodiments, the target pH is in a range of or of about 4 to 5. In some embodiments, the target pH is or is about 4.1, 4.2, 4.3, 4.4., 4.5, 4.6, or 4.7. In some embodiments, the target pH is in a range of or of about 4.2 to 4.7. In some embodiments, the target pH is in a range of or of about 4.2 to 4.7. In some embodiments, the target pH is or is about 4.6.

In some embodiments, the stored thermophilic fermented dairy composition has a pH in the range of or of about 3.8 to 5. In some embodiments, the stored thermophilic fermented dairy composition has a pH in the range of or of about 4 to 5. In some embodiments, the stored thermophilic fermented dairy composition has a pH of or of about 4.1, 4.2, 4.3, 4.4., 4.5, 4.6, or 4.7. In some embodiments, the stored thermophilic fermented dairy composition has a pH in the range of or of about 4.2 to 4.7. In some embodiments, the stored thermophilic fermented dairy composition has a pH of or of about 4.6.

In some embodiments, the pH of the stored thermophilic fermented dairy composition is measured, e.g., monitored, during storage. In some embodiments, the pH of the stored thermophilic fermented dairy composition is measured, e.g., monitored, at specific time points during storage. For example, a pH measurement may be taken every or every other day, every third, every fourth, every fifth, every sixth, every seventh, every eighth, every ninth, every tenth, every eleventh, or every twelfth, every thirteenth, or every fourteenth day in storage for the duration of storage. In some embodiments, the pH of the stored thermophilic fermented dairy composition is measured, e.g., monitored at about every 5 hours, 10 hours, 15 hours, 20 hours, 24 hours, 36 hours, 48 hours, 60 hours, or every 72 for the duration of time in storage. In some embodiments, the pH of the stored thermophilic fermented dairy composition is measured, e.g., monitored, continuously during storage. In some embodiments, the pH of the stored thermophilic fermented dairy composition is measured at a temperature of or of about 20° C. For example, the stored thermophilic fermented dairy composition may be removed from storage for a period of time to increase the temperature of the stored thermophilic fermented dairy composition prior to measuring the pH.

In some embodiments, the thermophilic fermented dairy composition, e.g., stored thermophilic fermented dairy composition, is stored for at least or at least about 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 40, 50, or 60 days. In some embodiments, the thermophilic fermented dairy composition, e.g., stored thermophilic fermented dairy composition, is stored for at least or at least about 7, 8, 9, 10, 11, 12, 13, 14 15, 16, 17, 18, 19, 20, 21, 22, 23, 24, 25, 26, 27, or 28 days. In some embodiments, the thermophilic fermented dairy composition, e.g., stored thermophilic fermented dairy composition, is stored for at least or at least about 60 days. In some embodiments, the thermophilic fermented dairy composition, e.g., stored thermophilic fermented dairy composition, is stored for at least or at least about 30 days. In some embodiments, the thermophilic fermented dairy composition, e.g., stored thermophilic fermented dairy composition, is stored for at least or at least about 28 days. In some embodiments, the thermophilic fermented dairy composition, e.g., stored thermophilic fermented dairy composition, is stored for at least or at least about 21 days. In some embodiments, the thermophilic fermented dairy composition, e.g., stored thermophilic fermented dairy composition, is stored for at least or at least about 14 days. In some embodiments, the thermophilic fermented dairy composition, e.g., stored thermophilic fermented dairy composition, is stored for at least or at least about 7 days.

In some embodiments, the thermophilic fermented dairy composition, e.g., stored thermophilic fermented dairy composition, is stored for at most or at most about 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 40, 50, or 60 days. In some embodiments, the thermophilic fermented dairy composition, e.g., stored thermophilic fermented dairy composition, is stored for at most or at most about 7, 8, 9, 10, 11, 12, 13, 14 15, 16, 17, 18, 19, 20, 21, 22, 23, 24, 25, 26, 27, or 28 days. In some embodiments, the thermophilic fermented dairy composition, e.g., stored thermophilic fermented dairy composition, is stored for at most or at most about 60 days. In some embodiments, the thermophilic fermented dairy composition, e.g., stored thermophilic fermented dairy composition, is stored for at most or at most about 30 days. In some embodiments, the thermophilic fermented dairy composition, e.g., stored thermophilic fermented dairy composition, is stored for at most or at most about 28 days. In some embodiments, the thermophilic fermented dairy composition, e.g., stored thermophilic fermented dairy composition, is stored for at most or at most about 21 days. In some embodiments, the thermophilic fermented dairy composition, e.g., stored thermophilic fermented dairy composition, is stored for at most or at most about 14 days. In some embodiments, the thermophilic fermented dairy composition, e.g., stored thermophilic fermented dairy composition, is stored for at most or at most about 7 days.

In some embodiments, the thermophilic fermented dairy composition, e.g., stored thermophilic fermented dairy composition, is stored for a duration of or of about 1 to 60, 1 to 55, 1 to 50, 1 to 45, 1 to 40, 1 to 35, 1 to 30, 1 to 25, 1 to 20, 1 to 15, 1 to 10, or 1 to 5 days. In some embodiments, the thermophilic fermented dairy composition, e.g., stored thermophilic fermented dairy composition, is stored for a duration of or of about 7 to 28, 7 to 21, or 7 to 14 days. In some embodiments, the thermophilic fermented dairy composition, e.g., stored thermophilic fermented dairy composition, is stored for a duration of or of about 7 to 21 or 7 to 14 days. In some embodiments, the thermophilic fermented dairy composition, e.g., stored thermophilic fermented dairy composition, is stored for a duration of or of about 28 days. In some embodiments, the thermophilic fermented dairy composition, e.g., stored thermophilic fermented dairy composition, is stored for a duration of or of about 21 days. In some embodiments, the thermophilic fermented dairy composition, e.g., stored thermophilic fermented dairy composition, is stored for a duration of or of about 14 days. In some embodiments, the thermophilic fermented dairy composition, e.g., stored thermophilic fermented dairy composition, is stored for a duration of or of about 7 days.

In some embodiments, the thermophilic fermented dairy composition, e.g., stored thermophilic fermented dairy composition, is stored for an amount of time or at least an amount of time suitable for the levels of acetaldehyde and diacetyl in the thermophilic fermented dairy composition, e.g., stored thermophilic fermented dairy composition, to reach a relative quantity or ratio correlated with a cream flavor, e.g., increased cream flavor. In some embodiments, the thermophilic fermented dairy composition, e.g., stored thermophilic fermented dairy composition, is stored until a relative quantity or ratio of acetaldehyde to diacetyl as described in Section I-E is reached. In some embodiments, the thermophilic fermented dairy composition, e.g., stored thermophilic fermented dairy composition, is stored until a cream flavor, e.g., increased cream flavor, as described in Section I-E is reached.

### E. Assessing Acetaldehyde, Diacetyl, and Cream Flavor

In some aspects, the thermophilic fermented dairy composition produced according to the methods provided herein has cream flavor, such as an increased cream flavor. In some embodiments, the cream flavor is a cream aroma and/or cream odor. Without being bound by theory, the cream flavor may be correlated with the relative quantities of diacetyl and acetaldehyde present in the thermophilic fermented dairy composition. Thus, in some embodiments, the methods provided herein include decreasing acetaldehyde and increasing diacetyl content in the thermophilic fermented dairy composition to levels correlated with the perception of cream flavor, e.g., increased levels of cream aroma and/or cream odor, in the thermophilic fermented dairy composition. In some embodiments, the levels of acetaldehyde and diacetyl achieved according to the methods provided herein produce an increased cream flavor, e.g., cream aroma and/or cream odor, compared to other methods of producing a thermophilic fermented dairy composition that do not result in a relative content of acetaldehyde and diacetyl as described herein. In some embodiments, the increased cream flavor is an increased cream aroma and/or increased cream odor, e.g., as compared to thermophilic fermented dairy compositions produced via alternative methods.

Methods for detecting and optionally quantifying levels of volatile organic compounds, such as acetaldehyde and diacetyl, include but are not limited to mass spectrometry, e.g., gas chromatograph-mass spectrometry (GC-MS), selected ion flow tube mass spectrometry (SIFT-MS), proton transfer reaction mass spectrometry (PTR-MS), ion-molecule reaction mass spectrometry (IMR-MS). In some embodiments, acetaldehyde and diacetyl in the thermophilic fermented dairy composition are detected and quantified using GC-MS.

Methods for determining organoleptic profiles, including cream flavor, e.g., cream aroma, cream odor, are known in the art, and include, for example, the use of trained panels of sensory assessors to perform quantitative descriptive analysis (QDA). In some embodiments, the trained sensory assessors evaluate descriptors using a linear scale, e.g., 7-point linear scale. In some embodiments, evaluated descriptors include or are those shown in Table 3 of Section IV (see, Example 1). In some embodiments, the descriptors evaluated include cream aroma and cream odor. In some embodiments, the QDA is performed blind, e.g., the assessors do not know the identity or methods of producing the assessed thermophilic fermented dairy composition.

In some embodiments, the methods of producing a thermophilic fermented dairy composition having cream flavor include decreasing acetaldehyde content and increasing diacetyl content in the thermophilic fermented dairy composition. In some embodiments, the decrease in acetaldehyde and the increase in diacetyl results in a ratio of acetaldehyde to diacetyl that correlates with a cream flavor, e.g., an increased cream flavor.

The diacetyl content may be considered to increase with respect to an initial diacetyl content. In some embodiments, the initial diacetyl content is the diacetyl content of the thermophilic fermented dairy composition at the end of the fermentation process. In some embodiments, the initial diacetyl content is the diacetyl content of the thermophilic fermented dairy composition at the initiation of storage, e.g., the first day in storage. Similarly, the acetaldehyde content may be considered to decrease with respect to an initial acetaldehyde content. In some embodiments, the initial acetaldehyde content is the acetaldehyde content of the thermophilic fermented dairy composition at the end of the fermentation process. In some embodiments, the initial acetaldehyde content is the acetaldehyde content of the thermophilic fermented dairy composition at the initiation of storage, e.g., the first day in storage.

In some embodiments, the method of producing a thermophilic fermented dairy composition having cream flavor includes decreasing a level of acetaldehyde in the thermophilic fermented dairy composition compared to the level of acetaldehyde, e.g., initial acetaldehyde content, present in the thermophilic fermented dairy composition produced by thermophilic fermentation of the inoculated milk substrate. In some embodiments, the method of producing a thermophilic fermented dairy composition having cream flavor includes increasing a level of diacetyl in the thermophilic fermented dairy composition compared to the level of diacetyl, e.g., initial diacetyl content, present in the thermophilic fermented dairy composition produced by thermophilic fermentation of the inoculated milk substrate. In some embodiments, the method of producing a thermophilic fermented dairy composition having cream flavor includes decreasing a level of acetaldehyde in the thermophilic fermented dairy composition compared to the level of acetaldehyde, e.g., initial acetaldehyde content, present in the thermophilic fermented dairy composition produced by thermophilic fermentation of the inoculated milk substrate and increasing a level of diacetyl in the thermophilic fermented dairy composition compared to the level of diacetyl, e.g., initial diacetyl content, present in the thermophilic fermented dairy composition produced by thermophilic fermentation of the inoculated milk substrate. In some embodiments, the method of producing a thermophilic fermented dairy composition having cream flavor includes decreasing a level of acetaldehyde in the thermophilic fermented dairy composition compared to the level of acetaldehyde, e.g., initial acetaldehyde content, present in the thermophilic fermented dairy composition produced by thermophilic fermentation of the inoculated milk substrate and/or increasing a level of diacetyl in the thermophilic fermented dairy composition compared to the level of diacetyl, e.g., initial diacetyl content, present in the thermophilic fermented dairy composition produced by thermophilic fermentation of the inoculated milk substrate to produce a ratio of acetaldehyde to diacetyl correlated with a cream flavor, e.g., increased cream flavor, as described herein.

In some embodiments, the diacetyl content is increased by a factor of at least 1.5, e.g., 2, 5, 6, 7, 8, 9, 10, 11, 12, 13, 14, or 15. In some embodiments, the diacetyl content is increased by a factor of at least 2. In some embodiments, the diacetyl content is increased by a factor in the range of or of about 1.5 to 20, 1.5 to 15, 1.5 to 10, 1.5 to 9, 1.5 to 8, 1.5 to 7, 1.5 to 6, 1.5 to 5, 1.5 to 4, 1.5 to 3, or 1.5 to 2. In some embodiments, the diacetyl content is increased by a factor in the range of or of about 1.5 to 15, 1.5 to 10 ppm, 1.5 to 9, 1.5 to 8, 1.5 to 7, or 1.5 to 6. In some embodiments, the diacetyl content is increased by a factor in the range of or of about 1.5 to 10. In some embodiments, the diacetyl content is increased by a factor of 1.5, 2, 3, 4, 5, 6, 7, 8, 9, 10, 11, 12, 13, 14, 15, or 20. In some embodiments, the diacetyl content is increased by a factor of or of about 1.5, 2, 3, 4, 5, 6, 7, 8, 9, 10, 11, 12, 13, 14, or 15. In some embodiments, the diacetyl content is increased by a factor of or of about 1.5, 2, 3, 4, or 5. In some embodiments, the diacetyl content is increased by a factor of or of about 1.5. In some embodiments, the diacetyl content is increased by a factor of or of about 2.

In some embodiments, the method of producing a thermophilic fermented dairy composition having cream flavor includes increasing the diacetyl content by a factor described herein.

In some embodiments, the diacetyl content is increased to or to at least 1 ppm, 2 ppm, 3 ppm, 4 ppm, 5 ppm, 6 ppm, 7 ppm, 8 ppm, 9 ppm, 10 ppm, 11 ppm, 12 ppm, 13 ppm, 14 ppm, or 15 ppm or more. In some embodiments, the diacetyl content is increased to or to at least 1.5 ppm. In some embodiments, the diacetyl content is increased to or to at least 2 ppm. In some embodiments, the diacetyl content is increased to or to at least 3 ppm. In some embodiments, the diacetyl content is increased to or to at least 4 ppm. In some embodiments, the diacetyl content is increased to or to at least 5 ppm. In some embodiments, the diacetyl content is increased to or to at least 6 ppm. In some embodiments, the diacetyl content is increased to or to at least 7 ppm.

In some embodiments, the diacetyl content is increased to or to at least 8 ppm. In some embodiments, the diacetyl content is increased to or to at least 9 ppm. In some embodiments, the diacetyl content is increased to or to at least 10 ppm. In some embodiments, the diacetyl content is increased to or to at least 15 ppm.

In some embodiments, the diacetyl content is increased to between or between about 1 ppm to 15 ppm, 1 ppm to 14 ppm, 1 ppm to 13 ppm, 1 ppm to 12 ppm, 1 ppm to 11 ppm, 1 ppm to 10 ppm, 1 ppm to 9 ppm, 1 ppm to 8 ppm, 1 ppm to 7 ppm, 1 ppm to 6 ppm, 1 ppm to 5 ppm, 1 ppm to 4 ppm, 1 ppm to 3 ppm, 1 ppm to 2 ppm, or 1 ppm to 1.5 ppm. In some embodiments, the diacetyl content is increased to between or between about 1.5 ppm to 15 ppm, 2 ppm to 15 ppm, 2 ppm to 15 ppm, 4 ppm to 15 ppm, 6 ppm to 15 ppm, 7 ppm to 15 ppm, 8 ppm to 15 ppm, 9 ppm to 15 ppm, 10 ppm to 15 ppm, 11 ppm to 15 ppm, 12 ppm to 15 ppm, 13 ppm to 15 ppm, 14 ppm to 15 ppm. In some embodiments, the diacetyl content is increased to between or between about 1 ppm to 7 ppm, 1 ppm to 6 ppm, 1 ppm to 5 ppm, 1 ppm to 4 ppm, 1 ppm to 3 ppm, 1 ppm to 2 ppm, or 1 ppm to 1.5 ppm. In some embodiments, the diacetyl content is increased to between or between about 1 ppm to 7 ppm. In some embodiments, the diacetyl content is increased between or between about 1.5 ppm to 7 ppm. In some embodiments, the diacetyl content is increased to or to about 1 ppm, about 2 ppm, about 3 ppm, about 4 ppm, about 5 ppm, about 6 ppm, about 7 ppm, about 8 ppm, about 9 ppm, about 10 ppm, about 11 ppm, about 12 ppm, about 13 ppm, about 14 ppm, or about 15 ppm.

In some embodiments, the methods of producing a thermophilic fermented dairy composition having cream flavor produce a diacetyl content of or of at least 1 ppm, 2 ppm, 3 ppm, 4 ppm, 5 ppm, 6 ppm, 7 ppm, 8 ppm, 9 ppm, 10 ppm, 11 ppm, 12 ppm, 13 ppm, 14 ppm, or 15 ppm or more. In some embodiments, the diacetyl content produced is or is at least 1.5 ppm. In some embodiments, the diacetyl content produced is or is at least 2 ppm. In some embodiments, the diacetyl content produced is or is at least 3 ppm. In some embodiments, the diacetyl content produced is or is at least 4 ppm. In some embodiments, the diacetyl content produced is or is at least 5 ppm. In some embodiments, the diacetyl content produced is or is at least 6 ppm. In some embodiments, the diacetyl content produced is or is at least 7 ppm. In some embodiments, the diacetyl content produced is or is at least 8 ppm. In some embodiments, the diacetyl content produced is or is at least 9 ppm. In some embodiments, the diacetyl content is produced is or is at least 10 ppm. In some embodiments, the diacetyl content produced is in the range of or of about 1 ppm to 15 ppm, 1 ppm to 14 ppm, 1 ppm to 13 ppm, 1 ppm to 12 ppm, 1 ppm to 11 ppm, 1 ppm to 10 ppm, 1 ppm to 9 ppm, 1 ppm to 8 ppm, 1 ppm to 7 ppm, 1 ppm to 6 ppm, 1 ppm to 5 ppm, 1 ppm to 4 ppm, 1 ppm to 3 ppm, 1 ppm to 2 ppm, or 1 ppm to 1.5 ppm. In some embodiments, the diacetyl content produced is in the range of or of about 1 ppm to 7 ppm, 1 ppm to 6 ppm, 1 ppm to 5 ppm, 1 ppm to 4 ppm, 1 ppm to 3 ppm, 1 ppm to 2 ppm, or 1 ppm to 1.5 ppm. In some embodiments, the diacetyl content produced is in the range of or of about 1 ppm to 7 ppm. In some embodiments, the diacetyl content produced is in the range of or of about 1.5 ppm to 7 ppm. In some embodiments, the diacetyl content produced is or is about 1 ppm, about 2 ppm, about 3 ppm, about 4 ppm, about 5 ppm, about 6 ppm, about 7 ppm, about 8 ppm, about 9 ppm, about 10 ppm, about 11 ppm, about 12 ppm, about 13 ppm, about 14 ppm, or about 15 ppm.

In some embodiments, the method of producing a thermophilic fermented dairy composition having cream flavor includes increasing the diacetyl content to an amount described herein.

In some embodiments, the acetaldehyde content is decreased by a factor of at least 1.5, e.g., 2, 3, 4, 5, 6, 7, 8, 9, 10, 11, 12, 13, 14, 15, 20, 30, 40, or 50. In some embodiments, the acetaldehyde content is decreased by a factor of at least 2. In some embodiments, the acetaldehyde content is decreased by a factor in the range of or of about 1.5 to 15, 1.5 to 10, 1.5 to 9, 1.5 to 8, 1.5 to 7, 1.5 to 6, 1.5 to 5, 1.5 to 4, 1.5 to 3, or 1.5 to 2. In some embodiments, the acetaldehyde content is decreased by a factor in the range of or of about 1.5 to 10 ppm, 1.5 to 9, 1.5 to 8, 1.5 to 7, or 1.5 to 6. In some embodiments, the acetaldehyde content is decreased by a factor in the range of or of about 2 to 10. In some embodiments, the acetaldehyde content is decreased by a factor of 1.5, 2, 3, 4, 5, 6, 7, 8, 9, 10, 11, 12, 13, 14, 15, or 20. In some embodiments, the acetaldehyde content is decreased by a factor of or of about 1.5, 2, 3, 4, 5, 6, 7, 8, 9, 10, 11, 12, 13, 14, or 15. In some embodiments, the acetaldehyde content is decreased by a factor of or of about 1.5, 2, 3, 4, or 5. In some embodiments, the acetaldehyde content is decreased by a factor of or of about 1.5. In some embodiments, the acetaldehyde content is decreased by a factor of or of about 2.

In some embodiments, the method of producing a thermophilic fermented dairy composition having cream flavor includes decreasing the acetaldehyde content by a factor described herein.

In some embodiments, the acetaldehyde content is decreased to or to less than 1 ppm, 0.5 ppm, 0.4 ppm, 0.3 ppm, 0.2 ppm, 0.1 ppm, 0.09 ppm, 0.08 ppm, 0.07 ppm, 0.06 ppm, 0.05 ppm, 0.03 ppm, 0.02 ppm, 0.01 ppm or less. In some embodiments, the acetaldehyde content is decreased to a range of or of about 0.5 ppm to about 0 ppm, e.g., below the lower limit of quantification and/or detection, 0.4 ppm to about 0 ppm, e.g., below the lower limit of quantification and/or detection, 0.3 ppm to about 0 ppm, e.g., below the lower limit of quantification and/or detection, 0.2 ppm to about 0 ppm, e.g., below the lower limit of quantification and/or detection, or 0.1 ppm to about 0 ppm, e.g., below the lower limit of quantification and/or detection. In some embodiments, the acetaldehyde content is decrease to about or below the lower limit of quantification and/or detection. In some embodiments, the acetaldehyde content is decreased to a range of or of about 0.2 ppm to about 0 ppm, e.g., below the lower limit of quantification and/or detection. In some embodiments, the acetaldehyde content in the thermophilic fermented dairy composition is negligible. In some embodiments, the lower limit of detection and/or quantification is specific to the instrument used to detect the compound, e.g., acetaldehyde. In some embodiments, acetaldehyde is detected and quantified using GC-MS. In some embodiments, the lower limit for quantification is 0.125 ppm.

In some embodiments, the methods of producing a thermophilic fermented dairy composition having cream flavor produce an acetaldehyde content that is or is less than, e.g., about, 1 ppm, 0.5 ppm, 0.4 ppm, 0.3 ppm, 0.2 ppm, 0.1 ppm, 0.09 ppm, 0.08 ppm, 0.07 ppm, 0.06 ppm, 0.05 ppm, 0.03 ppm, 0.02 ppm, 0.01 ppm. In some embodiments, the acetaldehyde content produced is in a range of or of about 0.5 ppm to about 0 ppm, e.g., below the lower limit of quantification and/or detection, 0.4 ppm to about 0 ppm, e.g., below the lower limit of quantification and/or detection, 0.3 ppm to about 0 ppm, e.g., below the lower limit of quantification and/or detection, 0.2 ppm to about 0 ppm, e.g., below the lower limit of quantification and/or detection, or 0.1 ppm to about 0 ppm, e.g., below the lower limit of quantification and/or detection. In some embodiments, the acetaldehyde content produced is, is about, or below the lower limit of quantification and/or detection. In some embodiments, the acetaldehyde content produced is in a range of or of about 0.2 ppm to about 0 ppm, e.g., below the lower limit of quantification and/or detection. In some embodiments, the acetaldehyde content produced in the thermophilic fermented dairy composition is negligible. In some embodiments, the lower limit of detection and/or quantification is specific to the instrument used to detect the compound, e.g., acetaldehyde. In some embodiments, acetaldehyde is detected and quantified using GC-MS. In some embodiments, the lower limit for quantification is 0.125 ppm.

In some embodiments, the method of producing a thermophilic fermented dairy composition having cream flavor includes decreasing the acetaldehyde content to an amount described herein.

In some embodiments, the methods of producing a thermophilic fermented dairy composition having cream flavor, e.g., increased cream flavor, result in a ratio of acetaldehyde to diacetyl that is associated with cream flavor, e.g., increased cream flavor. In some embodiments, the ratio of acetaldehyde to diacetyl in the thermophilic fermented dairy composition associated with cream flavor, e.g., increased cream flavor, is less than 1, e.g., about 0.9, about 0.8, about 0.7, about 0.6, about 0.5, about 0.4, about 0.3, about 0.2, about 0.1, about 0.08, about 0.07, about 0.06, about 0.05, about 0.04, about 0.03, about 0.02, or about 0.01, or less. In some embodiments, the ratio of acetaldehyde to diacetyl in the thermophilic fermented dairy composition associated with cream flavor, e.g., increased cream flavor, is less than or less than about 0.85, 0.75, 0.5, 0.25, 0.1, 0.075, 0.05, 0.025, or 0.01. In some embodiments, the ratio of acetaldehyde to diacetyl in the thermophilic fermented dairy composition associated with cream flavor, e.g., increased cream flavor, is less than or less than about 0.5. In some embodiments, the ratio of acetaldehyde to diacetyl in the thermophilic fermented dairy composition associated with cream flavor, e.g., increased cream flavor, is less than or less than about 0.25. In some embodiments, the ratio of acetaldehyde diacetyl in the thermophilic fermented dairy composition associated with cream flavor, e.g., increased cream flavor, is less than or less than about 0.25, 0.1, 0.075, 0.05, 0.025, or 0.01. In some embodiments, the ratio of acetaldehyde to diacetyl in the thermophilic fermented dairy composition associated with cream flavor, e.g., increased cream flavor, is in the range of or of about 0.5 to 0, 0.25 to 0, 0.1 to 0, 0.075 to 0, 0.05 to 0, 0.025 to 0, or 0.01 to 0. In some embodiments, the ratio of acetaldehyde to diacetyl in the thermophilic fermented dairy composition associated with cream flavor, e.g., increased cream flavor, is in the range of or of about 0.25 to 0, 0.1 to 0, 0.075 to 0, 0.05 to 0, 0.025 to 0, or 0.01 to 0. In some embodiments, the ratio of acetaldehyde to diacetyl in the thermophilic fermented dairy composition associated with cream flavor, e.g., increased cream flavor, is in the range of or of about 0.5 to 0. In some embodiments, the ratio of acetaldehyde to diacetyl in the thermophilic fermented dairy composition associated with cream flavor, e.g., increased cream flavor, is in the range of or of about 0.25 to 0. In some embodiments, the ratio of acetaldehyde to diacetyl in the thermophilic fermented dairy composition associated with cream flavor, e.g., increased cream flavor, is in the range of or of about 0.1 to 0. In some embodiments, the ratio of acetaldehyde to diacetyl in the thermophilic fermented dairy composition associated with cream flavor, e.g., increased cream flavor, is in the range of or of about 0.05 to 0. In some embodiments, the ratio of acetaldehyde to diacetyl in the thermophilic fermented dairy composition associated with cream flavor, e.g., increased cream flavor, is in the range of or of about 0.05 to 0, 0.025 to 0, or 0.01 to 0. In some embodiments, the ratio embodiments, the ratio of acetaldehyde to diacetyl in the thermophilic fermented dairy composition associated with cream flavor, e.g., increased cream flavor, is or is about 0, e.g., when the acetaldehyde content is at or below the lower limit of detection and/or quantification.

In some embodiments, the acetaldehyde content is decreased and the diacetyl content is increased to produce a ratio of acetaldehyde to diacetyl that is less than 1, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01, or 0. In some embodiments, the acetaldehyde content is decreased and the diacetyl content is increased to produce a ratio of acetaldehyde to diacetyl that is less than 1, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01, or 0 and the diacetyl content is increased to at least or at least about 1 ppm. In some embodiments, the acetaldehyde content is decreased and the diacetyl content is increased to produce a ratio of acetaldehyde to diacetyl that is less than 0.85, 0.75, 0.5, 0.25, 0.1, 0.075, 0.05, 0.025, 0.01 or is or is about 0, and the diacetyl content is increased to at least or at least about 1 ppm. In some embodiments, the acetaldehyde content is decreased and the diacetyl content is increased to produce a ratio of acetaldehyde to diacetyl that is less than 1, 0.9, 0.8, 0.7, 0.6, 0.5, 0.4, 0.3, 0.2, 0.1, 0.08, 0.07, 0.06, 0.05, 0.04, 0.03, 0.02, or 0.01, or 0, and the diacetyl content is increased to at least or at least about 1.5 ppm. In some embodiments, the acetaldehyde content is decreased and the diacetyl content is increased to produce a ratio of acetaldehyde to diacetyl that is less than 0.85, 0.75, 0.5, 0.25, 0.1, 0.075, 0.05, 0.025, 0.01 or is or is about 0, and the diacetyl content is increased to at least or at least about 1.5 ppm. In some embodiments, the acetaldehyde content is decreased and the diacetyl content is increased to produce a ratio of acetaldehyde to diacetyl that is less than 0.5, 0.25, 0.1, 0.075, 0.05, 0.025, 0.01 or is or is about 0, and the diacetyl content is increased to a level disclosed in any embodiment herein.

In some embodiments, the diacetyl content is increased to about 1 ppm, about 2 ppm, about 3 ppm, about 4 ppm, about 5 ppm, about 6 ppm, about 7 ppm, about 8 ppm, about 9 ppm, about 10 ppm, about 11 ppm, about 12 ppm, about 13 ppm, about 14 ppm, or about 15 ppm, and the acetaldehyde content is decreased to produce a ratio of acetaldehyde to diacetyl that is less than 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, or 0. In some embodiments, the diacetyl content is increased to about 1 ppm, about 2 ppm, about 3 ppm, about 4 ppm, about 5 ppm, about 6 ppm, about 7 ppm, and the acetaldehyde content is decreased to produce a ratio of acetaldehyde to diacetyl that is less than 0.75, 0.5, 0.25, 0.1, 0.075, 0.05, 0.025, 0.01, or 0. In some embodiments, the diacetyl content is increased to about 1 ppm, about 2 ppm, about 3 ppm, about 4 ppm, about 5 ppm, about 6 ppm, about 7 ppm, and the acetaldehyde content is decreased to produce a ratio of acetaldehyde to diacetyl that is less than 0.75, 0.5, 0.25, 0.1, 0.075, 0.05, 0.025, 0.01, or 0. In some embodiments, the acetaldehyde content is decreased to a level disclosed herein to achieve a ratio disclosed herein.

In some embodiments, the acetaldehyde content is decreased to below about 1 ppm, and the diacetyl content is increased to above or above about 1 ppm. In some embodiments, the acetaldehyde content is decreased to below about 0.2 ppm, and the diacetyl content is increased to above about 1 ppm. In some embodiments, the acetaldehyde content is decreased to about 0 ppm and the diacetyl content is increased to above or above about 1.5 ppm. In some embodiments, the acetaldehyde content is decreased to below about 1 ppm, and the diacetyl content is increased to above or above about 1.5 ppm. In some embodiments, the acetaldehyde content is decreased to below about 0.2 ppm, and the diacetyl content is increased to above about 1.5 ppm. In some embodiments, the acetaldehyde content is decreased to about 0 ppm and the diacetyl content is increased to above or above about 1.5 ppm. In some embodiments, the acetaldehyde content is decreased to any level disclosed herein and the diacetyl content is increased to any level disclosed herein to obtain a ratio of acetaldehyde to diacetyl disclosed herein, e.g., a ratio correlated with cream flavor, e.g., increased cream flavor.

In some embodiments the methods of producing a thermophilic fermented dairy composition having cream flavor, e.g., increased cream flavor, produces an acetaldehyde content that is below about 1 ppm, and a diacetyl content is above or above about 1 ppm. In some embodiments, the acetaldehyde content produced is below about 0.2 ppm, and the diacetyl content is produced is above about 1 ppm. In some embodiments, the acetaldehyde content produced is about 0 ppm and the diacetyl content produced is above or above about 1.5 ppm. In some embodiments, the acetaldehyde content produced is below about 1 ppm, and the diacetyl content produced is above or above about 1.5 ppm. In some embodiments, the acetaldehyde content produced is below about 0.2 ppm, and the diacetyl content produced is above about 1.5 ppm. In some embodiments, the acetaldehyde content produced is about 0 ppm and the diacetyl content produced is above or above about 1.5 ppm. In some embodiments, the acetaldehyde content produced is any level disclosed herein and the diacetyl content produced is any level disclosed herein to obtain a ratio of acetaldehyde to diacetyl disclosed herein, e.g., a ratio correlated with cream flavor, e.g., increased cream flavor.

In some embodiments, the cream flavor of the thermophilic fermented dairy composition having levels of acetaldehyde and diacetyl and/or a ratio thereof described herein has an increased cream aroma and/or increased cream odor compared to a thermophilic fermented dairy composition having an acetaldehyde content and a diacetyl content resulting in a ratio of acetaldehyde to diacetyl of about equal to or greater than 0.95. In some embodiments, the cream flavor of the thermophilic fermented dairy composition having levels of acetaldehyde and diacetyl or a ratio thereof described herein has an increased cream aroma and/or increased cream odor compared to a thermophilic fermented dairy composition having an acetaldehyde content and a diacetyl content resulting in a ratio of acetaldehyde to diacetyl of about equal to or greater than 1.

In some embodiments, the content or ratio described in this Section that associates with a cream flavor, e.g., increased cream flavor, is achieved at the end of thermophilic fermentation. In some embodiments, the content or ratio described in this Section that associates with a cream flavor, e.g., increased cream flavor, is achieved at a time point after the end of thermophilic fermentation. For example, the content or ratio described in this Section that associates with a cream flavor, e.g., increased cream flavor, is achieved during storage of the thermophilic fermented dairy composition. In some embodiments, the content or ratio described in this Section, which correlates with a cream flavor, e.g., increased cream flavor, is achieved in a stored thermophilic fermented dairy composition. For example, in some cases, the content or ratio described in this Section, which correlates with a cream flavor, e.g., increased cream flavor, is achieved during storage of the thermophilic fermented dairy composition, e.g., stored thermophilic fermented dairy composition according to any of the methods of storage described in Section I-D above.

In some embodiments, the content or ratio described in this Section of acetaldehyde to diacetyl associated with a cream flavor, e.g., increased cream flavor, is reached after more than 1 day of storage of the thermophilic fermented dairy composition. In some embodiments, content or ratio described in this Section of acetaldehyde to diacetyl associated with a cream flavor, e.g., increased cream flavor, is reached in or in about a range of 1 to 60, 1 to 55, 1 to 50, 1 to 45, 1 to 40, 1 to 35, 1 to 30, 1 to 25, 1 to 20, 1 to 15, 1 to 10, or 1 to 5 days of storage of the thermophilic fermented dairy composition. In some embodiments, the content or ratio described in this Section of acetaldehyde to diacetyl associated with a cream flavor, e.g., increased cream flavor, is reached in or in about a range of 1 to 28, 1 to 21, or 1 to 14 or 1 to 7 days of storage of the thermophilic fermented dairy composition.

In some embodiments, the content or ratio described in this Section of acetaldehyde to diacetyl associated with a cream flavor, e.g., increased cream flavor, is reached within or less than 28 days of storage of the thermophilic fermented dairy composition. In some embodiments, the content or ratio described in this Section of acetaldehyde to diacetyl associated with a cream flavor, e.g., increased cream flavor, is reached within or less than 21 days of storage of the thermophilic fermented dairy composition. In some embodiments, the content or ratio described in this Section of acetaldehyde to diacetyl associated with a cream flavor, e.g., increased cream flavor, is reached within or less than 14 days of storage of the thermophilic fermented dairy composition. In some embodiments, the content or ratio described in this Section of acetaldehyde to diacetyl associated with a cream flavor, e.g., increased cream flavor, is reached within or less than 7 days of storage of the thermophilic fermented dairy composition.

In some embodiments, the content or ratio described in this Section of acetaldehyde to diacetyl associated with a cream flavor, e.g., increased cream flavor, is reached in or in about 28 days of storage of the thermophilic fermented dairy composition. In some embodiments, the content or ratio described in this Section of acetaldehyde to diacetyl associated with a cream flavor, e.g., increased cream flavor, is reached in or in about 21 days of storage of the thermophilic fermented dairy composition. In some embodiments, the content or ratio described in this Section of acetaldehyde to diacetyl associated with a cream flavor, e.g., increased cream flavor, is reached in or in about than 14 days of storage of the thermophilic fermented dairy composition. In some embodiments, the content or ratio described in this Section of acetaldehyde to diacetyl associated with a cream flavor, e.g., increased cream flavor, is reached in or in about 7 days of storage of the thermophilic fermented dairy composition.

### F. Thermophilic Fermented Dairy Products

Also provided herein are thermophilic fermented dairy products obtained by any of the methods described herein and having any of the properties described herein that produce a cream flavor, such as an increased cream flavor. In some aspects, a thermophilic fermented dairy composition is a food product that is a thermophilic fermented dairy product.

In some embodiments, the methods provided herein to produce a thermophilic fermented dairy composition having cream flavor produce a thermophilic fermented dairy product. In some embodiments, the thermophilic fermented dairy product contains a ratio of acetaldehyde content to diacetyl content that is less than 1, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01, or is 0. In some embodiments, the thermophilic fermented dairy product contains a ratio of acetaldehyde content to diacetyl content that is less than 0.8, 0.7, 0.6, 0.5, 0.4, 0.3, 0.2, 0.1, 0.09, 0.08, 0.07, 0.06, 0.05, 0.04, 0.03, 0.02, or 0.01, or is 0. In some embodiments, the thermophilic fermented dairy product contains a ratio of acetaldehyde content to diacetyl content that is or is less than 0.8. In some embodiments, the thermophilic fermented dairy product contains a ratio of acetaldehyde content to diacetyl content that is or is less than 0.75. In some embodiments, the thermophilic fermented dairy product contains a ratio of acetaldehyde content to diacetyl content that is or is less than 0.7. In some embodiments, the thermophilic fermented dairy product contains a ratio of acetaldehyde content to diacetyl content that is or is less than 0.5. In some embodiments, the thermophilic fermented dairy product contains a ratio of acetaldehyde content to diacetyl content that is or is less than 0.2. In some embodiments, the thermophilic fermented dairy product contains a ratio of acetaldehyde content to diacetyl content that is or is less than 0.1. In some embodiments, the content or ratio of acetaldehyde to diacetyl in the thermophilic fermented dairy product is any ratio described in Section I-E associated with cream flavor, e.g., an increased cream flavor.

In some embodiments, the thermophilic fermented dairy product contains a Lacticaseibacillus rhamnosus strain that decreases acetaldehyde in the thermophilic fermented dairy product. In some embodiments, the thermophilic fermented dairy product contains a Lacticaseibacillus rhamnosus strain that increases diacetyl in the thermophilic fermented dairy product. In some embodiments, the thermophilic fermented dairy product contains a Lacticaseibacillus rhamnosus strain increases diacetyl and decreases acetaldehyde in the thermophilic fermented dairy product. In some embodiments, the Lacticaseibacillus rhamnosus strain that decreases acetaldehyde and increases diacetyl is the same strain. Alternatively, different Lacticaseibacillus rhamnosus strains may decrease acetaldehyde and increase diacetyl.

In some embodiments, the thermophilic fermented dairy product contains a Lacticaseibacillus rhamnosus strain DSM33650 or a mutant thereof as described herein. In some embodiments, the thermophilic fermented dairy product contains a Lacticaseibacillus rhamnosus strain DSM22193 or a mutant thereof as described herein. In some embodiments, the thermophilic fermented dairy product contains a Lacticaseibacillus rhamnosus strain DSM22876 or a mutant thereof as described herein. In some embodiments, the thermophilic fermented dairy product contains a Streptococcus thermophilus.

In some embodiments, the thermophilic fermented dairy product contains Streptococcus thermophilus strain DSM33651 or a mutant thereof as described herein and a Lacticaseibacillus rhamnosus strain DSM33650 or a mutant thereof as described herein. In some embodiments, the thermophilic fermented dairy product contains Streptococcus thermophilus strain DSM33849 or a mutant thereof as described herein and a Lacticaseibacillus rhamnosus strain DSM33650 or a mutant thereof as described herein. In some embodiments, the thermophilic fermented dairy product contains Streptococcus thermophilus strain DSM33829 or a mutant thereof as described herein and a Lacticaseibacillus rhamnosus strain DSM33650 or a mutant thereof as described herein. In some embodiments, the thermophilic fermented dairy product contains Streptococcus thermophilus strain DSM33651 or a mutant thereof as described herein and a Lacticaseibacillus rhamnosus strain DSM22876 or a mutant thereof as described herein. In some embodiments, the thermophilic fermented dairy product contains Streptococcus thermophilus strain DSM33849 or a mutant thereof as described herein and a Lacticaseibacillus rhamnosus strain DSM22876 or a mutant thereof as described herein. In some embodiments, the thermophilic fermented dairy product contains Streptococcus thermophilus strain DSM33829 or a mutant thereof as described herein and a Lacticaseibacillus rhamnosus strain DSM22876 or a mutant thereof as described herein. In some embodiments, the thermophilic fermented dairy product contains Streptococcus thermophilus strain DSM33651 or a mutant thereof as described herein and a Lacticaseibacillus rhamnosus strain DSM22193 or a mutant thereof as described herein. In some embodiments, the thermophilic fermented dairy product contains Streptococcus thermophilus strain DSM33849 or a mutant thereof as described herein and a Lacticaseibacillus rhamnosus strain DSM22193 or a mutant thereof as described herein. In some embodiments, the thermophilic fermented dairy product contains Streptococcus thermophilus strain DSM33829 or a mutant thereof as described herein and a Lacticaseibacillus rhamnosus strain DSM22193 or a mutant thereof as described herein.

Thermophilic fermented dairy products contemplated herein include, but are not limited to, a food, which includes, but is not limited to, dairy foods and dairy food-derived products.

The term “food” is used in a broad sense and includes feeds, foodstuffs, food ingredients, food supplements, and functional foods. Here, the term “food” is used in a broad sense—and covers food for humans as well as food for animals (i.e. a feed). In a preferred aspect, the food is for human consumption.

In some embodiments, the thermophilic fermented dairy product is a functional food. As used herein, the term “functional food” means a food which is capable of providing not only a nutritional effect and/or a taste satisfaction, but is also capable of delivering a further beneficial effect to consumer. Although there is no legal definition of a functional food, most of the parties with an interest in this area agree that there are foods marketed as having specific health effects.

In some embodiments, the bacterial strains or bacterial compositions, e.g., of the starter culture, described herein may be—or may be added to—a food ingredient, a food supplement, or a functional food.

In some embodiments, the food may be in the form of a solution or as a solid—depending on the use and/or the mode of application and/or the mode of administration.

In some embodiments, the bacterial strains or bacterial compositions, e.g., start cultures, described herein can be used in the preparation of food products such as one or more of thermophilic fermented dairy products.

By way of example, the bacterial strain or bacterial composition, e.g., starter culture, can be used as an ingredient to prepare milk drinks and lactic acid bacteria drinks, yogurt, and/or drinking yogurt.

In some embodiments, the thermophilic fermented dairy product is a stirred type thermophilic fermented dairy product. In some embodiments, the thermophilic fermented dairy product is a set-type dairy product. In some embodiments, the thermophilic fermented dairy product is a yogurt, a cream, a matured cream, a cheese, a fromage frais, a milk beverage, a processed cheese, a cream dessert, a cottage cheese, an infant milk, or a kefir. In some embodiments, the thermophilic dairy product is a milk beverage. Any dairy products that have been fermented by thermophilic fermentation, for example as described in Section I-C, are contemplated as thermophilic fermented dairy products.

### II. METHODS FOR IDENTIFYING BACTERIA CAPABLE OF PRODUCING THERMOPHILIC FERMENTED DAIRY COMPOSITIONS HAVING CREAM FLAVOR

Provided herein are methods of identifying bacteria and compositions thereof capable of producing thermophilic fermented dairy compositions, such as thermophilic fermented dairy products, having cream flavor, e.g., increased cream flavor. In some embodiments, the method for identifying bacteria and bacterial compositions capable of producing thermophilic fermented dairy compositions having cream flavor, e.g., increased cream flavor, includes fermenting a milk substrate as described herein inoculated with one or more bacteria and determining whether the one or more bacteria produce increases in diacetyl and/or decreases in acetaldehyde content in the thermophilic fermented dairy composition that produce ratios and/or levels of acetaldehyde and diacetyl that are associated, e.g., correlated, with cream flavor, e.g., increased cream flavor. In some cases, the one or more bacteria able to produce such a result are identified as capable of producing thermophilic diary composition having cream flavor, e.g., increased cream flavor. In some aspects, the methods of identifying such bacteria include quantifying acetaldehyde and diacetyl content in a thermophilic fermented dairy composition fermented in the presence of one or more bacteria. In some embodiments the thermophilic fermented dairy composition is produced, e.g., fermented and stored, according to the methods provided herein, with bacteria or bacterial compositions to be assessed for their ability to produce levels and ratios of acetaldehyde and diacetyl associated with cream flavor. In some embodiments, one or more bacteria capable of producing a thermophilic fermented dairy composition having cream flavor are identified.

In some embodiments, the method of identifying bacteria capable of producing thermophilic fermented dairy compositions having cream flavor as described herein includes determining the ratio of acetaldehyde to diacetyl in a thermophilic fermented dairy composition fermented in the presence of one or more bacteria. In some embodiments, the method of identifying includes quantifying acetaldehyde content and diacetyl content in a thermophilic fermented dairy composition fermented in the presence of one or more bacteria and stored following fermentation, for example as described herein. In some embodiments, the method of identifying includes determining the ratio of acetaldehyde to diacetyl in a thermophilic fermented dairy composition fermented in the presence of one or more bacteria and stored following fermentation, e.g., as described herein. In some embodiments, the method of identifying includes quantifying acetaldehyde content and diacetyl content in a stored thermophilic fermented dairy composition fermented in the presence of one or more bacteria. In some embodiments, the method of identifying includes determining the ratio of acetaldehyde to diacetyl in a stored thermophilic fermented dairy composition, as described herein, fermented in the presence of one or more bacteria. In some embodiments, the quantifying of content and/or determination of ratio as described herein takes place at different time points over the duration of storage. Thus, in some cases, the change in diacetyl and/or acetaldehyde content may be determined overtime. In some embodiments, the quantifying of content and/or determination of ratio as described herein takes place continuously over the duration of storage.

In some embodiments, if the one or more bacteria produce a decrease in acetaldehyde levels as described in Section I-E, the one or more bacteria are identified as capable of producing thermophilic fermented dairy compositions having cream flavor. In some embodiments, if the one or more bacteria produce a decrease in acetaldehyde and an increase in diacetyl levels as described in Section I-E, the one or more bacteria are identified as capable of producing thermophilic fermented dairy compositions having cream flavor. In some embodiments, if the one or more bacteria produce a ratio of acetaldehyde to diacetyl associated with cream flavor as described in Section I-E, the one or more bacteria are identified as capable of producing thermophilic fermented dairy compositions having cream flavor. In some embodiments, if the one or more bacteria produce a levels of acetaldehyde and/or diacetyl and/or a ratio of acetaldehyde to diacetyl associated with cream flavor as described in Section I-E, the one or more bacteria are identified as capable of producing thermophilic fermented dairy compositions having cream flavor.

In some embodiments, the bacteria identified as capable of producing thermophilic fermented dairy compositions having cream flavor produce a decrease in acetaldehyde levels as described in Section I-E. In some embodiments, the bacteria identified as capable of producing thermophilic fermented dairy compositions having cream flavor produce a decrease in acetaldehyde and an increase in diacetyl levels as described in Section I-E. In some embodiments, the bacteria identified as capable of producing thermophilic fermented dairy compositions having cream flavor produce a ratio of acetaldehyde to diacetyl associated with cream flavor as described in Section I-E. In some embodiments, the bacteria identified as capable of producing thermophilic fermented dairy compositions having cream flavor produce a levels of acetaldehyde and/or diacetyl and/or a ratio of acetaldehyde to diacetyl associated with cream flavor as described in Section I-E.

In some embodiments, the bacteria identified as capable of producing thermophilic fermented dairy compositions having cream flavor is or includes Lacticaseibacillus rhamnosus strains. In some embodiments, the bacteria identified as capable of producing thermophilic fermented dairy compositions having cream flavor is or includes Streptococcus thermophilus strains. In some embodiments, the bacteria identified as capable of producing thermophilic fermented dairy compositions having cream flavor is or includes Lacticaseibacillus rhamnosus strains and Streptococcus thermophilus strains.

In some aspects, one or more bacteria identified as capable of producing a thermophilic fermented dairy composition having cream flavor are contained in a composition. In some embodiments, the composition is a starter culture. In some embodiments, the starter culture further includes one or more bacteria described in Section I-B. In some embodiments, the composition is a thermophilic fermented dairy product. In some embodiments, the thermophilic fermented dairy product is any one of those described in Section I-F.

### III. KITS

Also provided are kits including the compositions containing bacteria and bacterial compositions, e.g., starter cultures, described herein, which may further include instructions for using the compositions, such as described herein. The kits described herein may also include other materials desirable from a commercial and user standpoint, including other buffers, diluents, filters, needles, syringes, and package inserts with instructions for performing any methods described herein. In some embodiments, the kits provided herein include bacteria and/or bacterial compositions capable of producing a thermophilic fermented dairy product having cream flavor, such as an increased cream flavor.

### IV. DEPOSIT AND EXPERT SOLUTION

The following deposit was made according to the Budapest treaty on the international recognition of the deposit of microorganisms for the purposes of patent procedure.
Lacticaseibacillus rhamnosus strain (DGCC1179) deposited under accession number DSM 33650 on Sep. 29, 2020, at the DSMZ [Deutsche Sammlung von Mikroorganismen und Zellkulturen GmbH, Inhoffenstrasse 7B, D-38124 Braunschweig—Germany].
Lacticaseibacillus rhamnosus strain (DGCC1460) deposited under accession number DSM22876 on Jun. 1, 2021, at the DSMZ [Deutsche Sammlung von Mikroorganismen und Zellkulturen GmbH, Inhoffenstrasse 7B, D-38124 Braunschweig—Germany].
Lacticaseibacillus rhamnosus strain (DGCC9913) deposited under accession number DSM22193 on Jan. 15, 2009, at the DSMZ [Deutsche Sammlung von Mikroorganismen und Zellkulturen GmbH, Inhoffenstrasse 7B, D-38124 Braunschweig—Germany].
Streptococcus thermophilus strain (DGCC11042) deposited under accession number DSM33651 on Sep. 29, 2020, at the DSMZ [Deutsche Sammlung von Mikroorganismen und Zellkulturen GmbH, Inhoffenstrasse 7B, D-38124 Braunschweig—Germany].
Streptococcus thermophilus strain (DGCC7693) deposited under accession number DSM33829 on Feb. 23, 2021, at the DSMZ [Deutsche Sammlung von Mikroorganismen und Zellkulturen GmbH, Inhoffenstrasse 7B, D-38124 Braunschweig—Germany].
Streptococcus thermophilus strain (DGCC12456) deposited under accession number DSM33849 on Apr. 20, 2021, at the DSMZ [Deutsche Sammlung von Mikroorganismen und Zellkulturen GmbH, Inhoffenstrasse 7B, D-38124 Braunschweig—Germany].

It is requested that the biological material shall be made available only by the issue of a sample to an expert nominated by the requester. In respect to those designations in which a European Patent is sought, a sample of the deposited microorganism will be made available until the publication of the mention of the grant of the European patent or until the date on which application has been refused or withdrawn or is deemed to be withdrawn, only by the issue of such a sample to an expert nominated by the person requesting the sample, and approved either i) by the Applicant and/or ii) by the European Patent Office, whichever applies (Rule 32 EPC)

### V. EXEMPLARY EMBODIMENTS

Among the provided embodiments are:
1. A method for producing a thermophilic fermented dairy composition having a cream flavor, the method comprising decreasing acetaldehyde content and increasing diacetyl content in the thermophilic fermented dairy composition.
2. A method for producing a stored thermophilic fermented dairy composition having a cream flavor, the method comprising:
   (a) storing a thermophilic fermented dairy composition to produce the stored thermophilic fermented dairy composition; and
   (b) decreasing acetaldehyde content and increasing diacetyl content in the thermophilic fermented dairy composition during storage.
3. The method of embodiment 1 or embodiment 2, wherein the method further comprises producing the thermophilic fermented dairy composition, the producing comprising fermenting a milk substrate inoculated with a starter culture to produce the thermophilic fermented dairy composition.
4. A method for producing a thermophilic fermented dairy composition having a cream flavor, the method comprising:
   (a) fermenting a milk substrate inoculated with a starter culture to produce a thermophilic fermented dairy composition; and
   (b) decreasing acetaldehyde content and increasing diacetyl content in the thermophilic fermented dairy composition.
5. The method of any one of embodiments 1, 3, or 4, comprising storing the thermophilic fermented dairy composition to produce a stored thermophilic fermented dairy composition.
6. The method of embodiment 5, wherein the acetaldehyde content is decreased and the diacetyl content is increased in the thermophilic fermented dairy composition during storage.
7. The method of any one of embodiments 1-6, wherein:
   (a) the acetaldehyde content is decreased and the diacetyl content is increased to produce a ratio of acetaldehyde to diacetyl that is less than 1, 0.75, 0.5, 0.25, 0.1, 0.075, 0.05, 0.025, or 0.01, or is 0; and
   (b) the diacetyl content is increased to at least 1 ppm.
8. The method of any one of embodiments 1-7, wherein:
   (a) the diacetyl content is increased to about 1 ppm, about 5 ppm, about 10 ppm, or about 15 ppm; and
   (b) the acetaldehyde content is decreased to produce a ratio of acetaldehyde to diacetyl that is less than about 1, 0.75, 0.5, 0.25, 0.1, 0.075, 0.05, 0.025, or 0.01, or is 0.
9. The method of any one of embodiments 1-8, wherein:
   (a) the acetaldehyde content is decreased to below about 1 ppm and the diacetyl content is increased to above about 1.5 ppm; or
   (b) the acetaldehyde content is decreased to below about 0.2 ppm, and the diacetyl content is increased to above about 5 ppm.
10. The method of any one of embodiments 3-9, wherein the starter culture comprises one or more lactic acid bacteria.
11. The method of any one of embodiments 3-10, wherein the starter culture comprises bacteria from the genus Lactococcus, Lactobacillus, Streptococcus, Lacticaseibacillus, Leuconostoc, Pediococcus, Bifidobacterium, or any combination of the foregoing.
12. The method of any one of embodiments 3-11, wherein the starter culture comprises one or more Lacticaseibacillus rhamnosus strains.
13. The method according to embodiment 12, wherein the one or more Lacticaseibacillus rhamnosus strains comprise or consist of:
   (a) the Lacticaseibacillus rhamnosus strain deposited under accession number DSM33650 at the DSMZ or a mutant thereof;
   (b) the Lacticaseibacillus rhamnosus strain deposited under accession number DSM22876 at the DSMZ or a mutant thereof; and/or
(c) the Lacticaseibacillus rhamnosus strain deposited under accession number DSM22193 at the DSMZ or a mutant thereof.
14. The method of any one of embodiments 3-13, wherein the starter culture comprises one or more Streptococcus thermophilus strains.
15. The method of embodiment 14, wherein the one or more Streptococcus thermophilus strains comprise or consist of:
   (a) the Streptococcus thermophilus strain deposited under accession number DSM33651 at the DSMZ or a mutant thereof;
   (b) the Streptococcus thermophilus strain deposited under accession number DSM33849 at the DSMZ or a mutant thereof; and/or
(c) the Streptococcus thermophilus strain deposited under accession number DSM33829 at the DSMZ or a mutant thereof.
16. The method of any one of embodiments 3-15, wherein the starter culture comprises or consists of:
   (a) the Lacticaseibacillus rhamnosus strain deposited under accession number DSM 33650 at the DSMZ or a mutant thereof and the Streptococcus thermophilus strain deposited under accession number DSM 33651 at the DSMZ or a mutant thereof;
   (b) the Lacticaseibacillus rhamnosus strain deposited under accession number DSM33650 at the DSMZ or a mutant thereof and the Streptococcus thermophilus strain deposited under accession number DSM33849 at the DSMZ or a mutant thereof;
   (c) the Lacticaseibacillus rhamnosus strain deposited under accession number DSM33650 at the DSMZ or a mutant thereof and the Streptococcus thermophilus strain deposited under accession number DSM33829 at the DSMZ or a mutant thereof;
   (d) the Lacticaseibacillus rhamnosus strain deposited under accession number DSM 22876 at the DSMZ or a mutant thereof and the Streptococcus thermophilus strain deposited under accession number DSM 33651 at the DSMZ or a mutant thereof;
   (e) the Lacticaseibacillus rhamnosus strain deposited under accession number DSM22876 at the DSMZ or a mutant thereof and the Streptococcus thermophilus strain deposited under accession number DSM33849 at the DSMZ or a mutant thereof;
   (f) the Lacticaseibacillus rhamnosus strain deposited under accession number DSM22876 at the DSMZ or a mutant thereof and the Streptococcus thermophilus strain deposited under accession number DSM33829 at the DSMZ or a mutant thereof;
   (g) the Lacticaseibacillus rhamnosus strain deposited under accession number DSM22193 at the DSMZ or a mutant thereof and the Streptococcus thermophilus strain deposited under accession number DSM 33651 at the DSMZ or a mutant thereof;
   (h) the Lacticaseibacillus rhamnosus strain deposited under accession number DSM22193 at the DSMZ or a mutant thereof and the Streptococcus thermophilus strain deposited under accession number DSM33849 at the DSMZ or a mutant thereof; or
   (i) the Lacticaseibacillus rhamnosus strain deposited under accession number DSM22193 at the DSMZ or a mutant thereof and the Streptococcus thermophilus strain deposited under accession number DSM33829 at the DSMZ or a mutant thereof.
17. The method of any one of embodiments 3-16, wherein the starter culture comprises one or more of a Lactobacillus delbrueckii subsp bulgaricus strain, a Lactobacillus acidophilus strain, a Bifidobacterium lactis strain, Limosilactobacillus fermentum strain, a Lacticaseibacillus paracasei strain, a Lactiplantibacillus plantarum strain, a Propionibacteria freudenreichii strain, a Pediococcus acidilactici strain, a Lactococcus cremoris subsp cremoris strain, or a Lactococcus lactis subsp lactis strain.
18. The method of any one of embodiments 3-17, wherein the milk substrate is fermented at a temperature above about 33° C., 35° C., 36° C., 37° C., 38° C., 39° C., 40° C., 41° C., 42° C., 43° C., 44° C., 45° C., or 50° C.
19. The method of any one of embodiments 1-18, wherein the thermophilic fermented dairy composition has a pH of about 3.8 to about 5, optionally a pH of about 4.2 to about 4.6.
20. The method of any one of embodiments 2 and 5-19, wherein the thermophilic fermented dairy composition is stored at about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C. or about 26° C.
21. The method according to any one of embodiments 2 and 5-20, wherein the thermophilic fermented dairy composition is stored for:
   (a) at least about 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 40, 50, or about 60 days; or
   (b) at most about 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 40, 50, or about 60 days.
22. The method of any one of embodiments 2 and 5-21, wherein the thermophilic fermented dairy composition is stored for about 28 days at about 6° C.
23. The method according to any one of embodiments 2 and 5-21, wherein the thermophilic fermented dairy composition is stored for about 14 days at about 6° C.
24. The method of any one of embodiments 2 and 5-21, wherein the stored thermophilic fermented dairy composition has a pH of about 3.8 to about 5, optionally a pH of about 4.5 or about 4.6.
25. The method of any one of embodiments 3-24, wherein the milk substrate is cow milk.
26. The method of any one of embodiments 1-25, wherein the cream flavor comprises a cream aroma and/or cream odor.
27. The method of any of embodiments 1-26, wherein and the cream flavor is an increased cream flavor compared to a cream flavor of a thermophilic fermented dairy composition having an acetaldehyde content and a diacetyl content resulting in a ratio of acetaldehyde to diacetyl of equal to or greater than 1.
28. The method of embodiment 27, wherein the increased cream flavor comprises an increased cream aroma and/or cream odor compared to a cream aroma and/or cream odor of a thermophilic fermented dairy composition having an acetaldehyde content and a diacetyl content resulting in a ratio of acetaldehyde to diacetyl of equal to or greater than 1.
29. A composition comprising a Lacticaseibacillus rhamnosus strain, wherein the Lacticaseibacillus rhamnosus strain decreases acetaldehyde and increases diacetyl in a thermophilic fermented dairy composition to produce a cream flavor.
30. The composition of embodiment 29, wherein the Lacticaseibacillus rhamnosus strain is:
   (a) the Lacticaseibacillus rhamnosus strain deposited under accession number DSM33650 at the DSMZ or a mutant thereof;
   (b) the Lacticaseibacillus rhamnosus strain deposited under accession number DSM22876 at the DSMZ or a mutant thereof; or
   (c) the Lacticaseibacillus rhamnosus strain deposited under accession number DSM22193 at the DSMZ or a mutant thereof. 31. The composition of embodiment 29 or embodiment 30, further comprising a Streptococcus thermophilus strain.
32. The composition of any one of embodiments 29-31, wherein the Streptococcus thermophilus strains is:
   (a) the Streptococcus thermophilus strain deposited under accession number DSM33651 at the DSMZ or a mutant thereof;
   (b) the Streptococcus thermophilus strain deposited under accession number DSM33849 at the DSMZ or a mutant thereof; and/or
   (c) the Streptococcus thermophilus strain deposited under accession number DSM33829 at the DSMZ or a mutant thereof.
33. The composition of embodiment 31 or embodiment 32, comprising or consisting of:
   (a) the Lacticaseibacillus rhamnosus strain deposited under accession number DSM 33650 at the DSMZ or a mutant thereof and the Streptococcus thermophilus strain deposited under accession number DSM 33651 at the DSMZ or a mutant thereof;
   (b) the Lacticaseibacillus rhamnosus strain deposited under accession number DSM33650 at the DSMZ or a mutant thereof and the Streptococcus thermophilus strain deposited under accession number DSM33849 at the DSMZ or a mutant thereof;
   (c) the Lacticaseibacillus rhamnosus strain deposited under accession number DSM33650 at the DSMZ or a mutant thereof and the Streptococcus thermophilus strain deposited under accession number DSM33829 at the DSMZ or a mutant thereof;
   (d) the Lacticaseibacillus rhamnosus strain deposited under accession number DSM 22876 at the DSMZ or a mutant thereof and the Streptococcus thermophilus strain deposited under accession number DSM 33651 at the DSMZ or a mutant thereof;
   (e) the Lacticaseibacillus rhamnosus strain deposited under accession number DSM22876 at the DSMZ or a mutant thereof and the Streptococcus thermophilus strain deposited under accession number DSM33849 at the DSMZ or a mutant thereof;
   (f) the Lacticaseibacillus rhamnosus strain deposited under accession number DSM22876 at the DSMZ or a mutant thereof and the Streptococcus thermophilus strain deposited under accession number DSM33829 at the DSMZ or a mutant thereof;
   (g) the Lacticaseibacillus rhamnosus strain deposited under accession number DSM 22193 at the DSMZ or a mutant thereof and the Streptococcus thermophilus strain deposited under accession number DSM 33651 at the DSMZ or a mutant thereof;
   (h) the Lacticaseibacillus rhamnosus strain deposited under accession number DSM22193 at the DSMZ or a mutant thereof and the Streptococcus thermophilus strain deposited under accession number DSM33849 at the DSMZ or a mutant thereof; or
   (i) the Lacticaseibacillus rhamnosus strain deposited under accession number DSM22193 at the DSMZ or a mutant thereof and the Streptococcus thermophilus strain deposited under accession number DSM33829 at the DSMZ or a mutant thereof.
34. The composition of any one of embodiments 29-33, further comprising one or more of a Lactobacillus delbrueckii subsp bulgaricus strain, a Lactobacillus acidophilus strain, a Bifidobacterium lactis strain, Limosilactobacillus fermentum strain, a Lacticaseibacillus paracasei strain, a Lactiplantibacillus plantarum strain, a Propionibacteria freudenreichii strain, a Pediococcus acidilactici strain, a Lactococcus cremoris, or a Lactococcus lactis strain.
35. The composition of any one of embodiments 29-34, wherein the composition is a starter culture or a thermophilic fermented dairy product.
36. Use of a Lacticaseibacillus rhamnosus to decrease acetaldehyde content and increase diacetyl content in a thermophilic fermented dairy composition to produce cream flavor.
37. The use of embodiment 36, wherein the Lacticaseibacillus rhamnosus is:
   (a) the Lacticaseibacillus rhamnosus strain deposited under accession number DSM33650 at the DSMZ or a mutant thereof;
   (b) the Lacticaseibacillus rhamnosus strain deposited under accession number DSM22876 at the DSMZ or a mutant thereof; or
   (c) a Lacticaseibacillus rhamnosus strain deposited under accession number DSM22193 at the DSMZ or a mutant thereof.
38. The composition of any one of embodiments 29-35 or the use of embodiment 36 or embodiment 37, wherein the acetaldehyde content is decreased and the diacetyl content is increased to produce a ratio of acetaldehyde to diacetyl that is less than 1, 0.75, 0.5, 0.25, 0.1, 0.075, 0.05, 0.025, or 0.01, or is 0.
39. The composition of any one of embodiments 29-35 and 38 or the use of embodiments 36-38, wherein the cream flavor comprises a cream aroma and/or cream odor.
40. The composition of any one of embodiments 29-35 and 37-39 or the use of any one of embodiments 36-39, wherein and the cream flavor is an increased cream flavor compared to a cream flavor of a thermophilic fermented dairy composition having an acetaldehyde content and a diacetyl content resulting in a ratio of acetaldehyde to diacetyl of equal to or greater than 1.
41. The composition of embodiment 39 or the use of embodiment 39, wherein the increased cream flavor comprises an increased cream aroma and/or cream odor compared to a cream aroma and/or cream odor of a thermophilic fermented dairy composition having an acetaldehyde content and a diacetyl content resulting in a ratio of acetaldehyde to diacetyl of equal to or greater than 1.
42. A thermophilic fermented dairy composition obtained by the method of any one of embodiments 1-28 or the use of any one of embodiments 36-41.
43. The thermophilic fermented dairy composition of embodiment 42, wherein the thermophilic fermented dairy composition is a thermophilic fermented dairy product.
44. The thermophilic fermented dairy composition of embodiment 42 or embodiment 43, wherein the thermophilic fermented dairy composition is a yogurt, a cream, a matured cream, a cheese, fromage frais, a milk beverage, a processed cheese, a cream dessert, a cottage cheese, an infant milk, or a kefir.
45. A thermophilic fermented dairy product having cream flavor, the thermophilic fermented dairy product comprising a ratio of acetaldehyde content to diacetyl content that is less than 1, 0.75, 0.5, 0.25, 0.1, 0.075, 0.05, 0.025, or 0.01, or is 0.
46. The thermophilic fermented dairy product having cream flavor of embodiment 45, wherein the thermophilic fermented dairy product comprises a Lacticaseibacillus rhamnosus strain that decreases acetaldehyde and increases diacetyl content in the thermophilic fermented dairy product.
47. The thermophilic fermented dairy product having cream flavor of embodiment 45 or embodiment 36, wherein the Lacticaseibacillus rhamnosus strain is:
   (a) a Lacticaseibacillus rhamnosus strain deposited under accession number DSM33650 or a mutant thereof;
   (b) a Lacticaseibacillus rhamnosus strain deposited under accession number DSM22876 or a mutant thereof; and/or
   (c) a Lacticaseibacillus rhamnosus strain deposited under accession number DSM22193 or a mutant thereof.
48. The thermophilic fermented dairy product having cream flavor of any one of embodiments 45-47, further comprising a Streptococcus thermophilus strain.
49. The thermophilic fermented dairy product having cream flavor of embodiment 48, wherein the Streptococcus thermophilus strain is:
   (a) the Streptococcus thermophilus strain deposited under accession number DSM33651 at the DSMZ or a mutant thereof;
   (b) the Streptococcus thermophilus strain deposited under accession number DSM33849 at the DSMZ or a mutant thereof; and/or
   (c) the Streptococcus thermophilus strain deposited under accession number DSM33829 at the DSMZ or a mutant thereof.
50. The thermophilic fermented dairy product of any one of embodiments 45-49, wherein the thermophilic fermented dairy product is a yogurt, a cream, a matured cream, a cheese, fromage frais, a milk beverage, a processed cheese, a cream dessert, a cottage cheese, an infant milk, or a kefir.
51. A method of identifying one or more bacteria capable of producing a thermophilic fermented dairy composition having cream flavor, the method comprising quantifying acetaldehyde content and diacetyl content in a thermophilic fermented dairy composition fermented in the presence of one or more bacteria.
52. The method of embodiment 51, wherein the thermophilic fermented dairy composition is stored and the acetaldehyde and diacetyl content are quantified during storage.
53. The method of embodiment 51 or 52, wherein if the quantified acetaldehyde content and diacetyl content produce a ratio of acetaldehyde content to diacetyl content in the thermophilic fermented dairy composition that is less than 1, 0.75, 0.5, 0.25, 0.1, 0.075, 0.05, 0.025, or 0.01, or is 0, the one or more bacteria are identified as capable of producing a thermophilic fermented dairy composition having cream flavor.
54. The method of any one of embodiments 51-53, wherein the one or more bacteria comprise lactic acid bacteria.
55. The method of any one of embodiments 51-54, wherein the one or more bacteria comprise a Lacticaseibacillus rhamnosus strain.
56. The method according to any one of embodiments 51-55, wherein the one or more bacteria comprise a Streptococcus thermophilus strain.
57. The method according to any one of embodiments 7-28 and 51-56, the composition of any one of embodiments 38-41, the use of any one of embodiments 38-41, the thermophilic fermented dairy composition of any one of embodiments 42-44, or the thermophilic fermented dairy product of any one of embodiments 45-50, wherein the ratio of acetaldehyde to diacetyl is reached after 28 days or less in storage.
58. The method according to any one of embodiments 7-28 and 53-57, the composition of any one of embodiments 38-41 and 57, the use of any one of embodiments 38-41 and 57, the thermophilic fermented dairy composition of any one of embodiments 42-44 and 57, or the thermophilic fermented dairy product of any one of embodiments 45-50 and 57, wherein the ratio of acetaldehyde to diacetyl is reached after 21 days or less in storage.
59. The method according to any one of embodiments 7-28 and 53-58, the composition of any one of embodiments 38-41, 57, and 58, the use of any one of embodiments 38-41, 57, and 58, the thermophilic fermented dairy composition of any one of embodiments 42-44, 57, and 58, or the thermophilic fermented dairy product of any one of embodiments 45-50, 57 and 58, wherein the ratio of acetaldehyde to diacetyl is reached after 14 days or less in storage.
60. The method according to any one of embodiments 7-28 and 53-56, the composition of any one of embodiments 38-41 and 57-59, the use of any one of embodiments 38-41 and 57-59, the thermophilic fermented dairy composition of any one of embodiments 42-44 and 57-59, or the thermophilic fermented dairy product of any one of embodiments 45-50 and 57-59, wherein the ratio of acetaldehyde to diacetyl is reached after 7 days or less in storage.
61. A composition comprising one or more bacteria identified as capable of producing a thermophilic fermented dairy composition having cream flavor according to the method of any one of embodiments 51-60.
62. The composition of embodiment 60, wherein the composition is a starter culture or a thermophilic fermented dairy product.
63. A kit comprising:
   (a) the composition of any one of embodiments 29-35, 38-41, and 57-62; and
   (b) instructions for use.
64. A Streptococcus thermophilus strain deposited under accession number DSM33849 at the DSMZ or a mutant thereof.
65. A Streptococcus thermophilus strain deposited under accession number DSM33829 at the DSMZ or a mutant thereof.
66. A Streptococcus thermophilus strain deposited under accession number DSM33651 at the DSMZ or a mutant thereof.
67. A Lacticaseibacillus rhamnosus strain deposited under accession number DSM22876 at the DSMZ or a mutant thereof.
68. A Lacticaseibacillus rhamnosus strain deposited under accession number DSM22193 at the DSMZ or a mutant thereof.

### VI. EXAMPLES

The following examples are included for illustrative purposes only and are not intended to limit the scope of the invention.

### Example 1: Assessment of Cream Flavor in Thermophilic Fermented Milk

The ability to increase cream flavor in a thermophilic fermented milk using bacterial compositions was assessed.

### A. Materials and Methods

### Milk Substrates

Two Candia Grandlait fresh milk (full fat) compositions were used as a milk substrate for thermophilic fermentation:
1. Plain full milk with 3.4% protein
2. Diluted milk with 3% protein (addition of 7% of water to plain full milk).

Both milks were heat treated at 90° C. for 10 minutes and then cooled to 43° C. for fermentation.

### Bacterial Compositions

Exemplary Streptococcus thermophilus strain DSM33849 was studied in combination with one of five exemplary Lacticaseibacillus rhamnosus (L. rhamnosus) strains: DSM22876, DSM33650, DSM22193, DSM24616 and an exemplary industrial strain Strain 5. The Streptococcus thermophilus (S. thermophilus) strain was also evaluated alone as control. In some cases, the combination of DSM33849 and DSM22876 was tested twice and the second fermented milk is denoted with “(2)” in the figures and Tables.

Bulk starter (BS) for each of the L. rhamnosus strains was prepared by inoculating 100 mL of reconstituted milk with 10% of SMP BBA Lactalis (heat treated 20 min at 120° C.) with a vial of the L. rhamnosus strain.

A working stock of DSM24616 was prepared by inoculating 100 mL of reconstituted milk containing 10% of SMP BBA Lactalis (heat treated 20 min at 120° C.). The inoculated composition was heated for 18 h at 37° C. before 100 mL of reconstituted milk with 10% of SMP BBA Lactalis (heat treated 20 min at 120° C.) was added and the mixture homogenized. After homogenization, the culture was aliquoted in 2 mL vials.

After 18-19 h of incubation at 37° C., 2% of each BS was used to inoculate the milk substrate together with 2 U/100 L of the S. thermophilus. The St. thermophilus was also inoculated at 2 U/100 L alone as a control.

Sensory Analysis with Trained Panel

Sensory analysis was performed on the thermophilic fermented milk after storage at 6° C. for 1 day and 14 days (2 replicates at 14 days).

A trained panel composed of 15-17 well-trained sensory assessors evaluated the thermophilic fermented milk using quantitative descriptive analysis (QDA). A total of 13 descriptors were evaluated on a linear 7-point scale (Table E1). The samples were randomly coded with 3-digit numbers and presented according to a Latin square. An ANOVA statistical test was used to assess significant differences between the samples on each descriptor of odor, texture, taste, aroma and perception (with p value<0.05 & <0.1).

TABLE E1
List of descriptors used for evaluating
thermophilic fermented milk.

| | |
|---|---|
| ODOR | FUNDAMENTAL TASTE |
| Odor Intensity | Acid |
| Cream odor | Sweet |
| SPOON TEXTURE | Bitter |
| Ropiness | AROMA & |
| Smoothness | PERCEPTION |
| MOUTH TEXTURE | Aroma Intensity |
| Mouth thickness | Cream aroma |
| Coating | Astringent |
| | Pungent |

### Volatile Organic Compounds

Volatile organic compound analysis was performed on milk before fermentation (TO) and after storage at 6° C. for 1, 14, and 28 days.

Volatile organic compounds were analyzed by gas chromatography-mass spectrometry (GC-MS) (ThermoFisher Scientific, USA; TRACE1300 GC coupled with an ISQ mass detector).

Samples were prepared in 10 mL head-space vials by combining 1 g of NaCl, 2 g of thermophilic fermented milk, 1 mL of MilliQ cooled water, and 100 μl of internal standard (solution of butylacetate at 25 ppm). Samples were vortexed prior to placement on the refrigerated autosampler of the gas chromatogram.

Volatile organic compounds were extracted from samples after heating (80° C. for 15 min) and shaking, by ITEX (dynamic headspace). The ITEX Trap was then desorbed in the GC-MS injector at 200° C. Molecules were separated on a ZB-Wax (60 m×0.25 mm×0.25 μm) column from Phenomenex with helium as carrier gas at 1 mL/min.

Volatile organic compounds were ionized by electronic impact (70 eV) and detected by a quadrupole mass spectrometer in full scan mode from 30 to 200 amu.

Key molecules of interest, including diacetyl and acetaldehyde, were quantified by external calibration with correction using an internal standard. Results are expressed in ppm.

### B. Results

### i) Full Milk Substrate

### Sensory Analysis

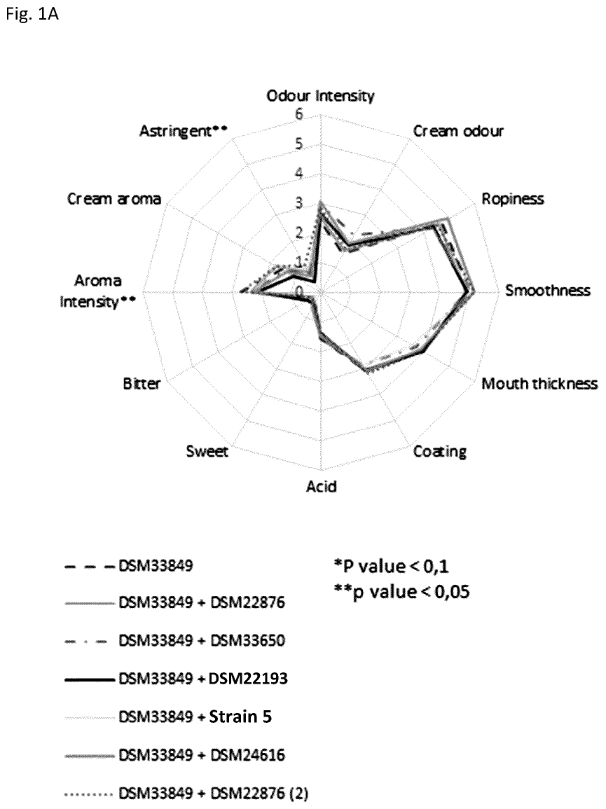

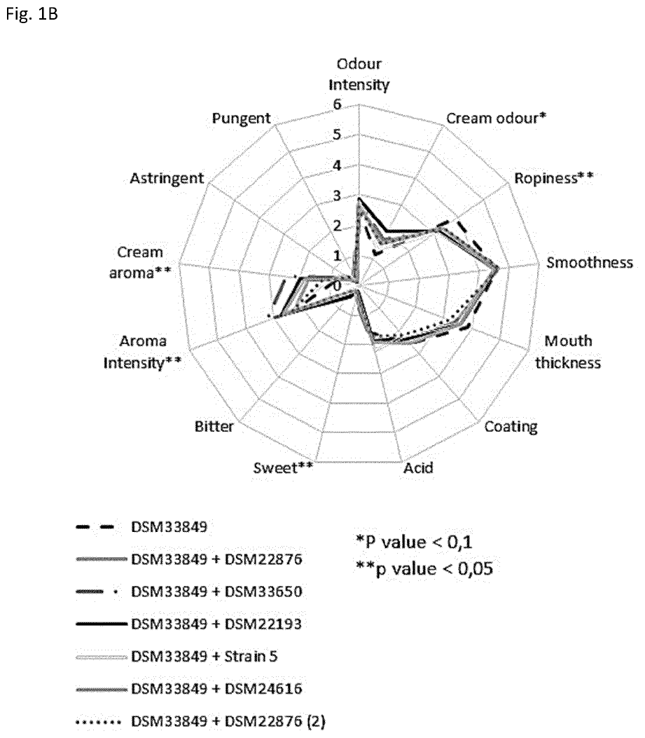

Organoleptic profiles of the thermophilic fermented milk were determined after 1 and 14 days in storage at 6° C. by a trained panel of assessors as described above. As shown in FIG. 1A, organoleptic profiles between the seven fermented milks were similar after 1 day in storage. Cream aroma was similar across the seven samples (FIG. 1A).

As shown in FIG. 1B, at day 14 in storage, cream odor and cream aroma differed across the seven samples. The sample containing DSM33650 had a higher value for cream aroma compared to the other samples (see, FIG. 1B and Table E2).

TABLE E2
Mean values for the cream aroma
descriptor after 14 days in full milk.

| Strain Combinations | Cream Aroma* |
|---|---|
| DSM33849 (Control) | 0.84 (E) |
| Strain 5 + DSM33849 | 1.12 (DE) |
| DSM22876 (2) + DSM33849 | 1.26 (CDE) |
| DSM22876 + DSM33849 | 1.67 (BCD) |
| DSM24616 + DSM33849 | 1.78 (BC) |
| DSM22193 + DSM33849 | 1.96 (AB) |
| DSM33650 + DSM33849 | 2.43 (A) |

*Letters shared between groups indicate the groups are not significantly different.

### Volatile Organic Compounds

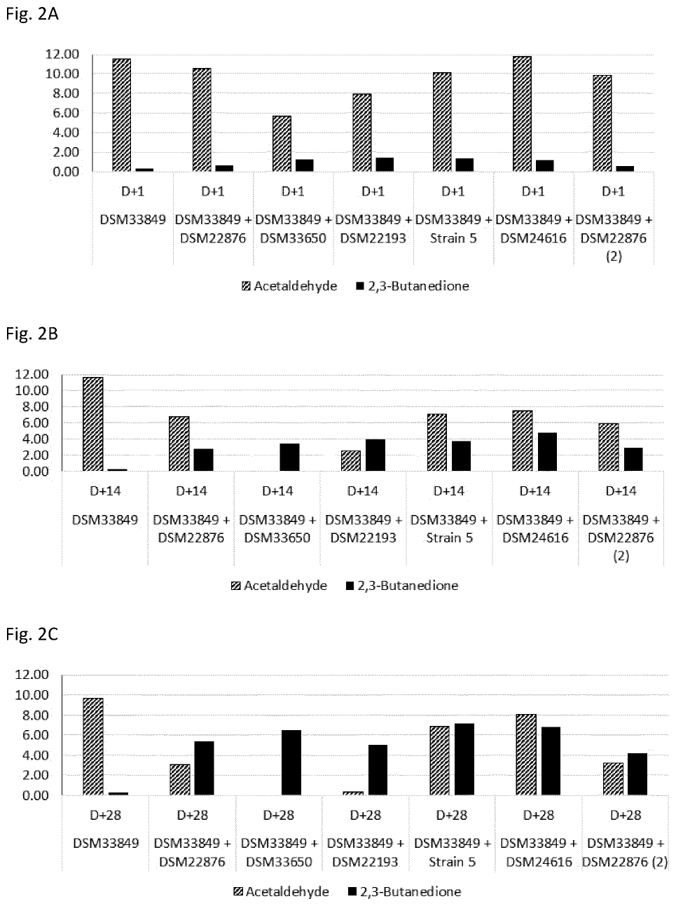

Volatile organic compounds were detected using GC-MS as described above at different time points during storage of the thermophilic fermented milk at 6° C. Diacetyl (2,3-butanedione) and acetaldehyde were found to be the main volatile organic compounds to evolve during storage (FIGS. 2A-2C).

FIG. 2A shows diacetyl and acetaldehyde content after 1 day of storage at 6° C. All milks fermented with S. thermophilus and L. rhamnosus strains showed an increased diacetyl content compared to the control fermented milk (S. thermophilus alone). The level of acetaldehyde was decreased in a subset of the milks fermented with S. thermophilus and L. rhamnosus strains compared to the control fermented milk (S. thermophilus alone). Milks fermented with S. thermophilus and L. rhamnosus strains DSM33650 or DSM22193 showed the greatest difference in acetaldehyde content compared to control. The acetaldehyde content of the milk fermented with DSM33650 was about half the amount observed in the control fermented milk. Both milks fermented with S. thermophilus and L. rhamnosus strain DSM22876 showed similar levels of diacetyl and acetaldehyde.

FIG. 2B shows diacetyl and acetaldehyde content in the fermented milks after 14 days of storage at 6° C. All experimental samples containing L. rhamnosus strains showed increased diacetyl content compared to the control fermented milk. The level of acetaldehyde was decreased in milks fermented with S. thermophilus and L. rhamnosus strains DSM33650 or DSM22193 compared to control, with the level of acetaldehyde in the fermented milk containing DSM33650 approaching the lower level of detection and/or quantification (0.125 ppm). Milks fermented with S. thermophilus and L. rhamnosus strains DSM22876, Strain 5, or DSM24616 showed a decreased level of acetaldehyde compared to control, although the difference was smaller than for samples containing strains DSM33650 or DSM22193.

FIG. 2C shows diacetyl and acetaldehyde content in the fermented milks after 28 days of storage at 6° C. All milks fermented with L. rhamnosus strains showed an increased diacetyl content compared to the control fermented milk. The level of acetaldehyde declined further for milks fermented with S. thermophilus and L. rhamnosus strains DSM33650, DSM22193, or DSM22876. The level of acetaldehyde in milks fermented with S. thermophilus and L. rhamnosus strains DSM24616 or Strain 5 was higher compared to the other fermented milks containing L. rhamnosus strains, and the levels of acetaldehyde appeared to be maintained from day 14 to 28.

These results are supportive of a correlation between an increased diacetyl content and a decreased acetaldehyde content in thermophilic fermented plain full milk and an increase in cream flavor, e.g., cream aroma and cream odor. These results suggest that cream flavor, e.g., cream aroma, cream odor, is increased in thermophilic fermented milks having a higher diacetyl content than acetaldehyde content.

### ii) Diluted Milk Substrate

### Sensory Analysis

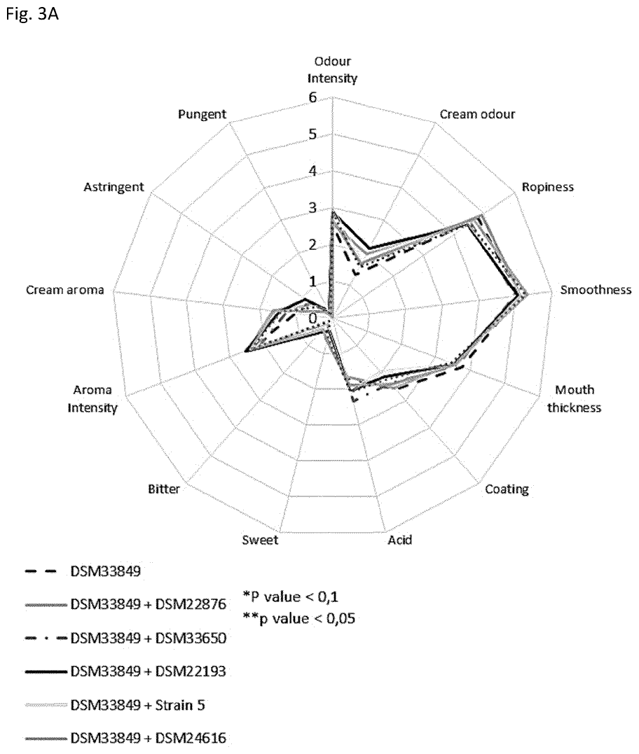

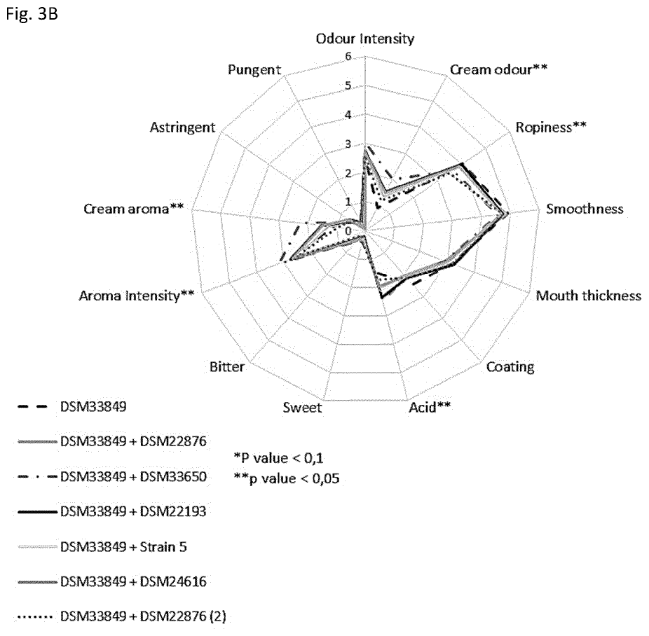

Organoleptic profiles of the thermophilic fermented diluted milk were determined after 1 and 14 days in storage at 6° C. by a trained panel of sensory assessors as described above. As shown in FIG. 3A, organoleptic profiles between the seven fermented milks were relatively similar after 1 day in storage. There were no observed differences in cream aroma between the seven samples (FIG. 3A).

As shown in FIG. 3B, on day 14 in storage, the milk fermented with S. thermophilus in combination with L. rhamnosus strain DSM33650 showed higher values for cream aroma and cream odor compared to the other samples (see, Table E3).

TABLE E3
Mean values for the cream aroma descriptor
after 14 days in diluted milk.

| Strains | Cream Aroma* |
|---|---|
| DSM22876 (2) + DSM33849 | 0.96 (B) |
| DSM33849 (Control) | 1.13 (B) |
| Strain 5 + DSM33849 | 1.28 (B) |
| DSM22876 + DSM33849 | 1.29 (B) |
| DSM24616 + DSM33849 | 1.49 (B) |
| DSM22193 + DSM33849 | 1.52 (B) |
| DSM33650 + DSM33849 | 2.24 (A) |

*Letters shared between groups indicate the groups are not significantly different.

### Volatile Organic Compounds

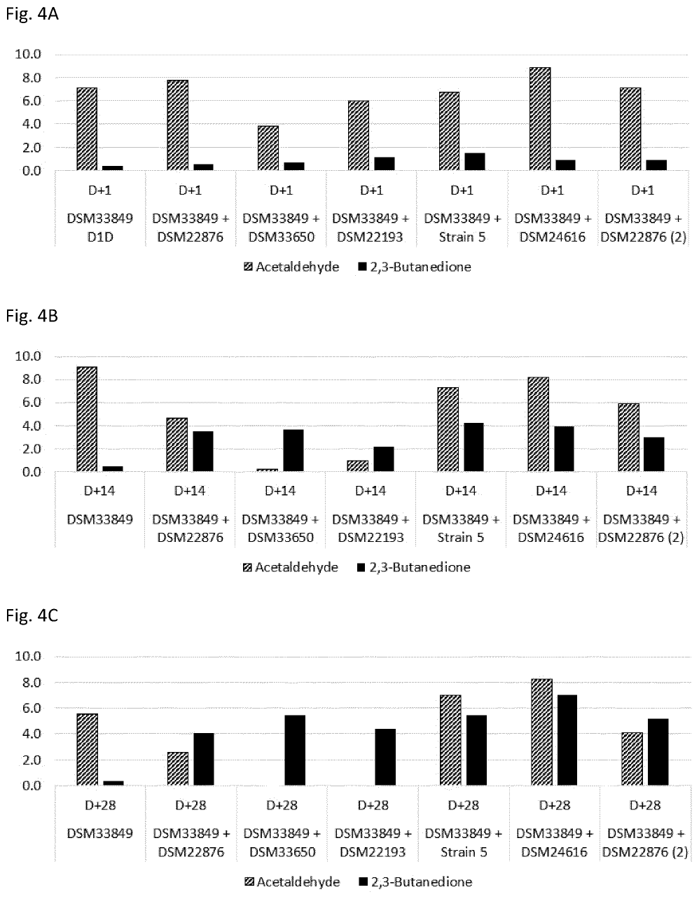

Volatile organic compounds were detected using GC-MS as described above at different time points during storage of the thermophilic fermented diluted milk at 6° C. Consistent with the plain full milk experiments, diacetyl (2,3-butanedione) and acetaldehyde were found to be the main volatile organic compounds that evolved during storage (FIGS. 4A-4C).

FIG. 4A shows diacetyl and acetaldehyde content in the fermented diluted milks after 1 day in storage at 6° C. The majority of milks fermented with S. thermophilus and L. rhamnosus strains showed an increased diacetyl content compared to the control fermented diluted milk (S. thermophilus alone). The level of acetaldehyde varied across the fermented diluted milks, the fermented milk containing L. rhamnosus strain DSM33650 having one of the lowest levels of acetaldehyde compared to control.

FIG. 4B shows diacetyl and acetaldehyde content in the fermented diluted milks after 14 days in storage at 6° C. All fermented milks containing L. rhamnosus strains showed increased diacetyl content compared to the control fermented diluted milk. The level of acetaldehyde was decreased in diluted milks fermented with S. thermophilus and L. rhamnosus strains DSM33650 or DSM22193 compared to control, with the level of acetaldehyde in the fermented milk containing strain DSM33650 approaching the lower level of detection and/or quantification (0.125 ppm). Diluted milks fermented with S. thermophilus and L. rhamnosus strains DSM22876, Strain 5, or DSM24616 showed decreased acetaldehyde content compared to control, although the difference was smaller than for fermented diluted milks containing DSM33650 or DSM22193.

After 28 days in storage at 6° C., all samples containing L. rhamnosus strains showed an increased diacetyl content compared to the control fermented diluted milk (FIG. 4C). The level of acetaldehyde was still declining for the diluted milks fermented with S. thermophilus and L. rhamnosus strains DSM22876, DSM33650, or DSM22193. The level of acetaldehyde in diluted milks fermented with S. thermophilus and L. rhamnosus strains Strain 5 or DSM24616 was higher compared to the other fermented milks containing L. rhamnosus strains, and the levels of acetaldehyde appeared to be maintained from day 14 to 28.

These results are supportive of a correlation between an increased diacetyl content and a decreased acetaldehyde content in thermophilic fermented plain diluted milk and an increase in cream flavor, e.g., cream aroma and cream odor. These results further suggest that cream flavor, e.g., cream aroma, cream odor, is increased in thermophilic fermented diluted milk having a higher diacetyl content than acetaldehyde content.

iii) Comparison of Volatile Compound Composition Between Full and Diluted Fermented Milk

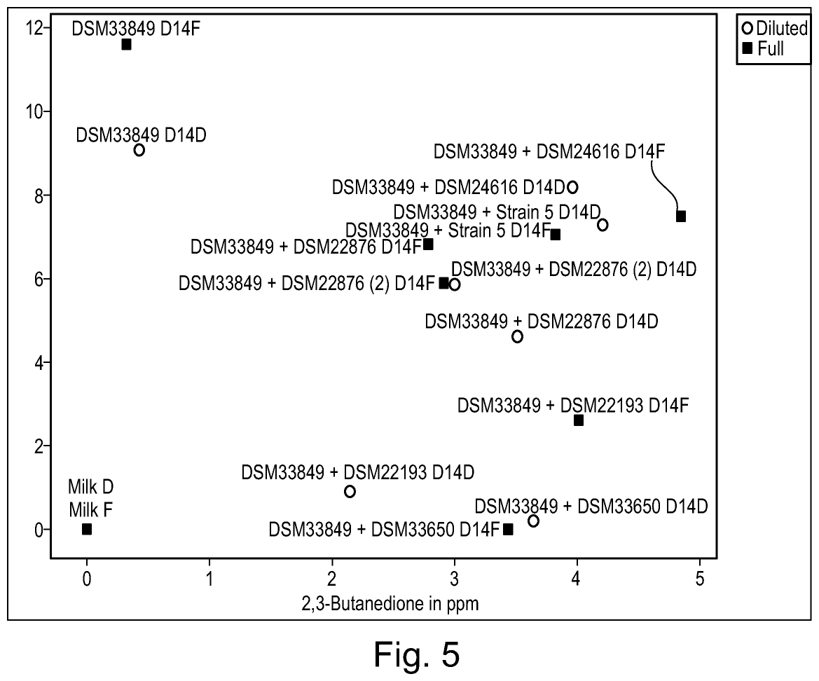

FIG. 5 shows a scatterplot of diacetyl (2, 3-butanedione) and acetaldehyde content for each of the tested full and diluted thermophilic fermented milks after 14 days in storage at 6° C. It can be seen in FIG. 5 that fermentations including L. rhamnosus have different volatile compositions compared to the control sample, and even between L. rhamnosus containing samples. For example, control fermented milks had low levels of diacetyl and high level of acetaldehyde, whereas samples including exemplary L. rhamnosus strain DSM33650 had a higher content of diacetyl and a low level of acetaldehyde.

While all samples containing L. rhamnosus strains showed increased levels of diacetyl compared to control, a subset of samples including L. rhamnosus strains also showed decreases in acetaldehyde content compared to control or lower levels of acetaldehyde than diacetyl. Initial diacetyl and acetaldehyde content in the milks prior to fermentation was close to zero or undetectable, e.g., below a lower limit of detection and/or quantification, which suggests that the changes in diacetyl and acetaldehyde relate to the fermentation process.

### C. Summary

The results observed for both full and diluted thermophilic fermented milks support a correlation between a combined increase in levels of diacetyl (known for imparting a cream/butter aroma) and decrease in levels of acetaldehyde (known for imparting a green/yogurt aroma) and an increase in cream flavor, e.g., cream odor and cream aroma. The results further support that thermophilic fermented milk having higher levels of diacetyl than acetaldehyde have increased cream flavor compared to thermophilic fermented milk with similar levels of acetaldehyde and diacetyl or where acetaldehyde levels are greater than diacetyl. These results also suggest that the inclusion of L. rhamnosus strains during fermentation can facilitate changes in these volatile compounds to impart cream flavor.

### Example 2: Assessment of Different Streptococcus thermophilus Strains in Combination with Lacticaseibacillus rhamnosus on Cream Flavor in Thermophilic Fermented Milk

The contribution of Streptococcus thermophilus (S. thermophilus) strains to the increase in cream flavor of thermophilic fermented full fat milk, as described in Example 1, was assessed.

Exemplary S. thermophilus strains DSM33849, DSM33651, and DSM33829 were studied in combination with one of two Lacticaseibacillus rhamnosus strains: DSM33650 or DSM24616. Each S. thermophilus strain was also evaluated alone as control.

Plain full milk was inoculated, fermented, stored, and analyzed generally as described in Example 1 above.

### A. Results

### Volatile Organic Compounds and Sensory Analysis

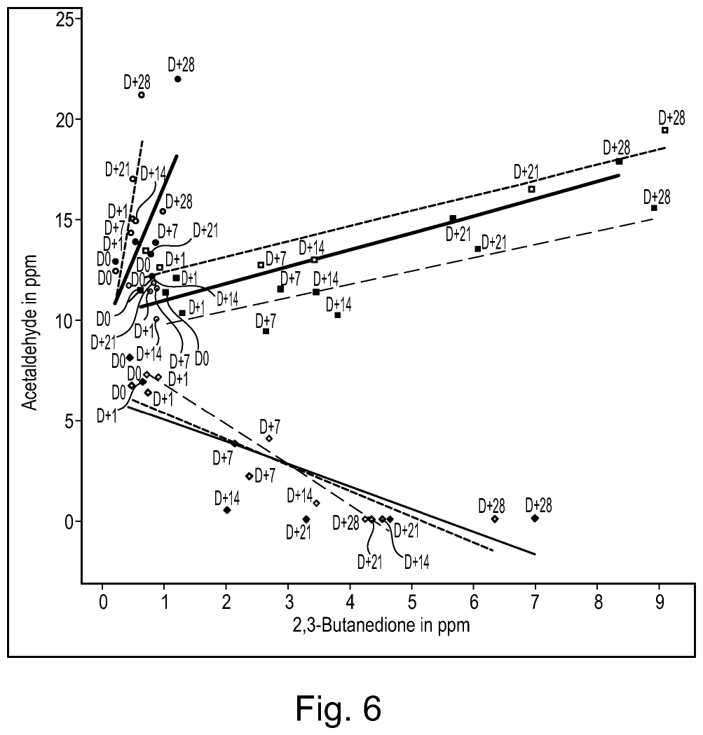

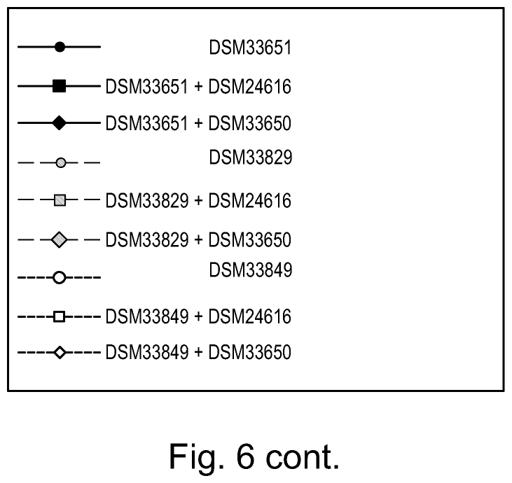

Volatile organic compounds were detected in the thermophilic fermented milks after 1, 7, 14, 21, and 28 days of storage at 6° C. As shown in FIG. 6, increases in diacetyl (2,3-butanedione) content and decreases in acetaldehyde content were observed over time in storage for milks fermented with L. rhamnosus strain DSM33650 regardless of S. thermophilus strain used, while increases in both diacetyl and acetaldehyde content were observed for milks fermented with DSM24616 regardless of S. thermophilus strain used. S. thermophilus control samples showed increases in acetaldehyde and minimal changes in diacetyl during storage.

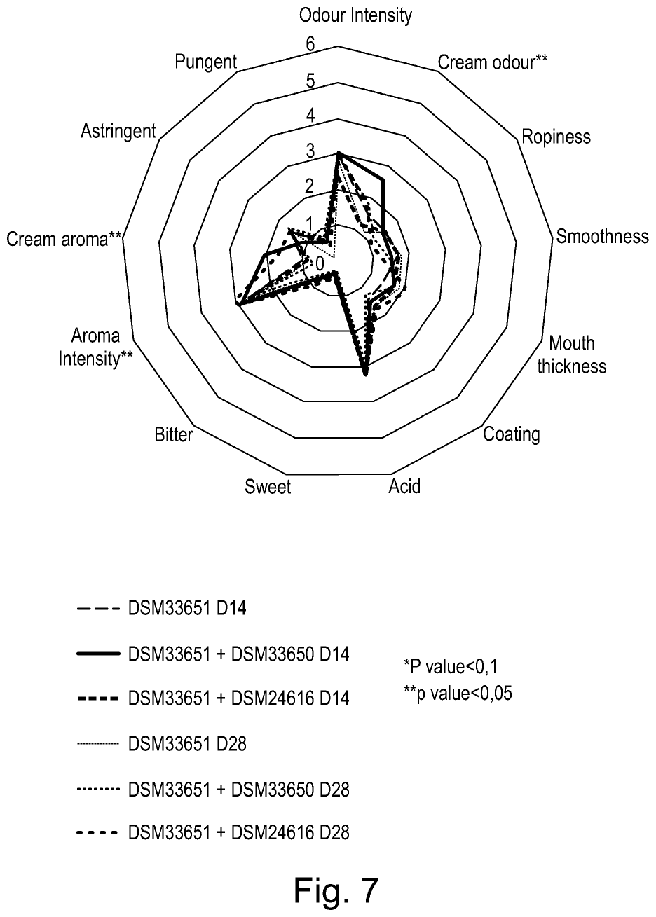

Organoleptic profiles of milks fermented with S. thermophilus stain DSM33651 alone or in combination with one of the exemplary L. rhamnosus strains were determined after 14 and 28 days in storage at 6° C. by a trained panel of assessors. As shown in FIG. 7, milks fermented with L. rhamnosus strain DSM33650 in combination with S. thermophilus DSM33651 showed an increased cream aroma on days 14 and 28 compared to the other samples.

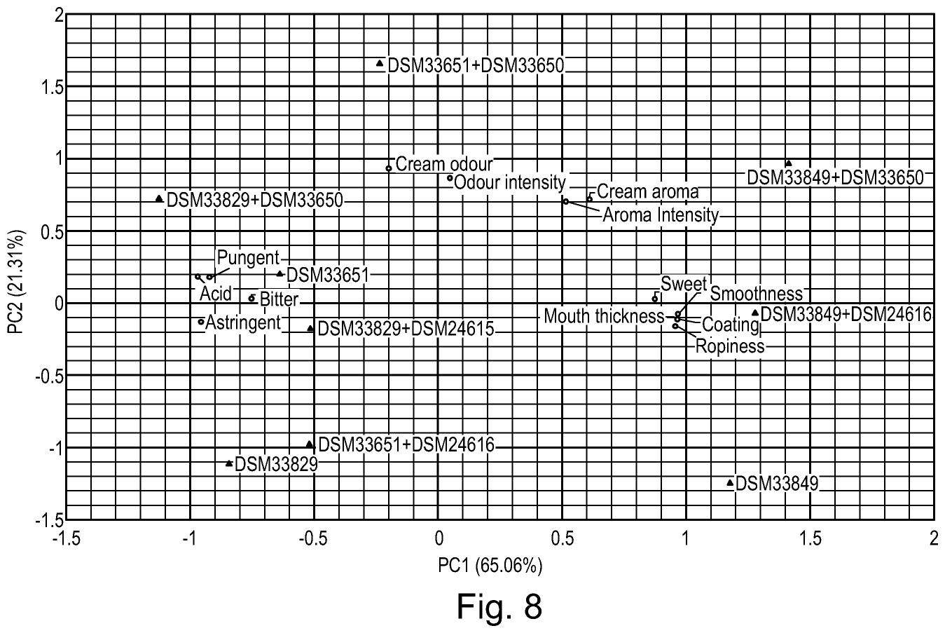

Organoleptic profiles determined on day 14 in storage were analyzed using Principal Component Analysis (PCA). In the PCA biplot shown in FIG. 8, L. rhamnosus strain DSM33650 in combination with any S. thermophilus strain clustered with cream aroma, aroma intensity (the overall aroma perception in mouth, including cream aroma), cream odor, and odor intensity (the overall odor perception with the nose, including cream odor). In addition, the perception of cream flavor persisted regardless of changes in perceived texture and acidity, e.g., lower texture and higher acidity, respectively.

### B. Summary

These results support a correlation between a combined increase in levels of diacetyl (known to impart a cream/butter aroma) and decrease in levels of acetaldehyde (known to impart a green/yogurt aroma) and cream flavor, e.g., cream odor and cream aroma, independent of changes in texture and/or acidity. The results further support that thermophilic fermented milk having higher levels of diacetyl than acetaldehyde have increased cream flavor compared to thermophilic fermented milk with similar levels of acetaldehyde and diacetyl or where acetaldehyde levels are greater than diacetyl. These results also suggest that the inclusion of L. rhamnosus strains during fermentation can facilitate changes in these volatile compounds to increase cream flavor in the thermophilic fermented milk independent of the S. thermophilus strain used in the fermentation process.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. Although the invention may be described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for producing a thermophilic fermented dairy composition having a cream flavor, the method comprising:
(a) fermenting at a temperature above about 33° C., 35° C., 36° C., 37° C., 38° C., 39° C., 40° C., 41° C., 42° C., 43° C., 44° C., 45° C., or 50° C. a milk substrate, optionally a cow milk, inoculated with a starter culture to produce a thermophilic fermented dairy composition; and
(b) decreasing an acetaldehyde content and increasing a diacetyl content in the thermophilic fermented dairy composition.

2. The method of claim 1, comprising storing the thermophilic fermented dairy composition.

3. The method of claim 2, wherein the acetaldehyde content is decreased and the diacetyl content is increased in the thermophilic fermented dairy composition during storage.

4. The method of any one of claims 1-3, wherein:
(a) the acetaldehyde content is decreased and the diacetyl content is increased to produce a ratio of acetaldehyde to diacetyl that is less than 1, 0.75, 0.5, 0.25, 0.1, 0.075, 0.05, 0.025, or 0.01, or is 0, and the diacetyl content is increased to at least 1 ppm; or
(b) the diacetyl content is increased to about 1 ppm, about 5 ppm, about 10 ppm, or about 15 ppm and the acetaldehyde content is decreased to produce a ratio of acetaldehyde to diacetyl that is less than about 1, 0.75, 0.5, 0.25, 0.1, 0.075, 0.05, 0.025, or 0.01, or is 0.

5. The method of any one of claims 1-4, wherein the starter culture comprises one or more Lacticaseibacillus rhamnosus strains, optionally wherein the one or more Lacticaseibacillus rhamnosus strains comprise or consist of:
(a) the Lacticaseibacillus rhamnosus strain deposited under accession number DSM33650 at the DSMZ or a mutant thereof;
(b) the Lacticaseibacillus rhamnosus strain deposited under accession number DSM22876 at the DSMZ or a mutant thereof; and/or
(c) the Lacticaseibacillus rhamnosus strain deposited under accession number DSM22193 at the DSMZ or a mutant thereof.

6. The method of any one of claims 1-5, wherein the starter culture comprises one or more Streptococcus thermophilus strains, optionally wherein the one or more Streptococcus thermophilus strains comprise or consist of:
(a) the Streptococcus thermophilus strain deposited under accession number DSM33651 at the DSMZ or a mutant thereof;
(b) the Streptococcus thermophilus strain deposited under accession number DSM33849 at the DSMZ or a mutant thereof; and/or
(c) the Streptococcus thermophilus strain deposited under accession number DSM33829 at the DSMZ or a mutant thereof.

7. The method of any one of claims 1-6, wherein the starter culture comprises or consists of:
(a) the Lacticaseibacillus rhamnosus strain deposited under accession number DSM33650 at the DSMZ or a mutant thereof and the Streptococcus thermophilus strain deposited under accession number DSM33651 at the DSMZ or a mutant thereof;
(b) the Lacticaseibacillus rhamnosus strain deposited under accession number DSM33650 at the DSMZ or a mutant thereof and the Streptococcus thermophilus strain deposited under accession number DSM33849 at the DSMZ or a mutant thereof;
(c) the Lacticaseibacillus rhamnosus strain deposited under accession number DSM33650 at the DSMZ or a mutant thereof and the Streptococcus thermophilus strain deposited under accession number DSM33829 at the DSMZ or a mutant thereof;
(d) the Lacticaseibacillus rhamnosus strain deposited under accession number DSM22876 at the DSMZ or a mutant thereof and the Streptococcus thermophilus strain deposited under accession number DSM33651 at the DSMZ or a mutant thereof;
(e) the Lacticaseibacillus rhamnosus strain deposited under accession number DSM22876 at the DSMZ or a mutant thereof and the Streptococcus thermophilus strain deposited under accession number DSM33849 at the DSMZ or a mutant thereof;
(f) the Lacticaseibacillus rhamnosus strain deposited under accession number DSM22876 at the DSMZ or a mutant thereof and the Streptococcus thermophilus strain deposited under accession number DSM33829 at the DSMZ or a mutant thereof;
(g) the Lacticaseibacillus rhamnosus strain deposited under accession number DSM22193 at the DSMZ or a mutant thereof and the Streptococcus thermophilus strain deposited under accession number DSM33651 at the DSMZ or a mutant thereof;
(h) the Lacticaseibacillus rhamnosus strain deposited under accession number DSM22193 at the DSMZ or a mutant thereof and the Streptococcus thermophilus strain deposited under accession number DSM33849 at the DSMZ or a mutant thereof; or
(i) the Lacticaseibacillus rhamnosus strain deposited under accession number DSM22193 at the DSMZ or a mutant thereof and the Streptococcus thermophilus strain deposited under accession number DSM33829 at the DSMZ or a mutant thereof.

8. The method of any one of claims 1-7, wherein the thermophilic fermented dairy composition:
(a) is stored at about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C. or about 26° C.; and
(b) is stored for at least about 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 40, 50, or about 60 days; or
(c) is stored for at most about 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 40, 50, or about 60 days.

9. A composition comprising a Lacticaseibacillus rhamnosus strain, wherein the Lacticaseibacillus rhamnosus strain decreases acetaldehyde and increases diacetyl in a thermophilic fermented dairy composition to produce a cream flavor, optionally wherein the acetaldehyde is decreased and the diacetyl is increased to produce a ratio of acetaldehyde to diacetyl that is less than 1, 0.75, 0.5, 0.25, 0.1, 0.075, 0.05, 0.025, or 0.01, or is 0.

10. Use of a Lacticaseibacillus rhamnosus to decrease acetaldehyde content and increase diacetyl content in a thermophilic fermented dairy composition to produce cream flavor; optionally wherein the acetaldehyde is decreased and the diacetyl is increased to produce a ratio of acetaldehyde to diacetyl that is less than 1, 0.75, 0.5, 0.25, 0.1, 0.075, 0.05, 0.025, or 0.01, or is 0.

11. A thermophilic fermented dairy composition obtained by the method of any one of claims 1-8 or the use of claim 10, optionally wherein the thermophilic fermented dairy composition is a thermophilic fermented dairy product, optionally a yogurt, a cream, a matured cream, a cheese, fromage frais, a milk beverage, a processed cheese, a cream dessert, a cottage cheese, an infant milk, or a kefir.

12. A thermophilic fermented dairy product having cream flavor, the thermophilic fermented dairy product comprising a ratio of acetaldehyde content to diacetyl content that is less than 1, 0.75, 0.5, 0.25, 0.1, 0.075, 0.05, 0.025, or 0.01, or is 0, optionally wherein the thermophilic fermented dairy product is a yogurt, a cream, a matured cream, a cheese, fromage frais, a milk beverage, a processed cheese, a cream dessert, a cottage cheese, an infant milk, or a kefir.

13. A method of identifying bacteria capable of producing a thermophilic fermented dairy composition having cream flavor, the method comprising quantifying acetaldehyde content and diacetyl content in a thermophilic fermented dairy composition fermented in the presence of one or more bacteria, optionally wherein the thermophilic fermented dairy composition is stored and the acetaldehyde and diacetyl content are quantified during storage; and
if the quantified acetaldehyde content and diacetyl content produce a ratio of acetaldehyde content to diacetyl content in the thermophilic fermented dairy composition that is less than 1, 0.75, 0.5, 0.25, 0.1, 0.075, 0.05, 0.025, or 0.01, or is 0, the one or more bacteria are identified as capable of producing a thermophilic fermented dairy composition having cream flavor.

14. A composition comprising one or more bacteria identified as capable of producing a thermophilic fermented dairy composition having cream flavor according to the method of claim 13, optionally wherein the composition is a starter culture or a thermophilic fermented dairy product.

15. A bacterial strain, wherein the strain is:
(a) a Streptococcus thermophilus strain deposited under accession number DSM33849 at the DSMZ or a mutant thereof;
(b) a Streptococcus thermophilus strain deposited under accession number DSM33829 at the DSMZ or a mutant thereof; or
(c) a Streptococcus thermophilus strain deposited under accession number DSM33651 at the DSMZ or a mutant thereof.
